(19) United States
(12) Patent Application Publication
(10) Pub. No.: US 2026/0201347 A1
(43) Pub. Date: Jul. 16, 2026

(54) ARTIFICIAL BIFUNCTIONAL ENZYME COMPRISING A MYELOPEROXIDASE ACTIVITY AND A GLUCOSE OXIDASE ACTIVITY AND APPLICATIONS THEREOF

(71) Applicants: UNIVERSITÉ DE BORDEAUX, BORDEAUX (FR); CENTRE NATIONAL DE LA RECHERCHE SCIENTIFIQUE, PARIS (FR)

(72) Inventors: CLAIRE STINES-CHAUMEIL, BORDEAUX (FR); CLAIRE CERE, BORDEAUX (FR); BRIGITTE DELORD, BORDEAUX (FR)

(21) Appl. No.: 19/137,324

(22) PCT Filed: Dec. 13, 2023
(86) PCT No.: PCT/EP2023/085555

§ 371 Date: Jun. 10, 2025

(30) Foreign Application Priority Data
Dec. 14, 2022 (EP) 22306877.6

(51) Int. Cl. C12N 9/08; A01N 63/50; A01P 1/00; A61K 38/00; A61P 31/04; C12N 9/04

(52) U.S. Cl. CPC C12N 9/0065; A01N 63/50; A01P 1/00; A61P 31/04; C12N 9/0006; C12Y 101/03004; C12Y 111/02002; A61K 38/00; C07K 2319/00

(57) ABSTRACT

The present invention relates to the field of enzymology. More particularly, the present invention relates to an artificial bifunctional enzyme, and applications thereof.

### INTRODUCTION

The present invention relates to the field of enzymology. More particularly, the present invention relates to an artificial bifunctional enzyme, and applications thereof.

Heme peroxidases are heme-containing enzymes found in all living organisms, which are capable to catalyze the formation of antimicrobial compounds and to participate in innate immunity. These peroxidases are divided into two main superfamilies: a first family found in plants, fungi and bacteria, which has likely arisen from gene duplication of a single common ancestral gene; and a second family found in mammals, which differs from the first family by its primary and tertiary structures as well as by its prosthetic group. Regardless of their origin, heme peroxidases can display a microbicidal activity thanks to their capacity to halogenate, in presence of hydrogen peroxide, a broad range of organic compounds which can be useful in biomedical, biotechnological or in the food industry. As an illustrative example, heme peroxidases can be a valuable therapy against bacterial infections, such as those that are resistant to antibiotics. That is because these enzymes are capable of catalyzing halides or pseudo-halides into (pseudo)hypohalous acids which are known to display potent bactericidal and antiviral activities.

Among heme peroxidases, mammalian peroxidases (MMPs) have been found to play a major role in the destruction of invading pathogens by the innate immune system. To this day, four main types of mammalian peroxidases have been discovered: the myeloperoxidase (MPO) expressed in neutrophils, the eosinophil peroxidase (EPO) localized in eosinophil granulocytes, the lactoperoxidase (LPO) expressed in mammary, salivary and other mucosal glands, and the thyroid peroxidase (TPO) found in the thyroid gland. The architecture of the active site of heme peroxidase is highly conserved. The heme is maintained in the protein via two covalent bonds, by autocatalytic formation of two ester bonds with an aspartate and glutamate residue. The myeloperoxidase (MPO) has a third covalent sulfonium bond, which provides a singular spectroscopic property (Soret band at 428 nm), thereby differentiating it from its counterparts. This bond is notably responsible for the chlorination activity of MPOs.

In nature, peroxidases are commonly found co-expressed with oxidases, simply because oxidases produce hydrogen peroxide—the substrate for peroxidases. A typical example can be found in fungi, where peroxidases that aid in biomass degradation are secreted along with oxidases which produce hydrogen peroxide, thereby fueling the peroxidases (Abdel-Hamid et al., Adv. Appl. Microbiol. 2013, 82: 1-28; Ander et al., J. Biotechnol. 1997, 53: 115-131).

From an industrial perspective, a wide range of applications have been implemented based on a combination of a peroxidase with an oxidase.

For example, the E-101 solution, essentially containing porcine MPO along with a glucose oxidase and sodium chloride, becomes microbicidal upon contact with glucose, thereby allowing the disinfection of human or veterinary injuries (Denys et al. Infect Immun., 2019; 87(7):e00261-19). The LPO has been identified as a suitable additive for preserving food such as milk, or as an oral disinfectant, when combined in a composition with glucose oxidase (WO2008105113A1; WO2011116052). The hMPO, together with a glucose oxidase, glucose and a halide, has also been reported as virucidal against HIV (Moguilevsky et al. FEBS Lett. 1992; 302(3):209-212).

Assays and biosensors based on the combinations of such enzymes have also been developed, so as to apply their complementary activities for measuring e.g. the concentration of glucose or uric acid in human serum samples (Barham et al., Analyst 1972, 97: 142-145; Chun et al., Biochip J. 2014, 8: 218-226; Mundaca-Uribe et al., Sens. Actuators B 2014, 195, 58-62).

These types of artificial systems, where the enzymes are free from each other, can however be relatively complex to implement, since they require identifying the experimental conditions that are optimal for both enzymes. In the case of assays and biosensors, the co-immobilization of the two enzymes on a biocompatible surface is also necessary to ensure their sufficient spatial closeness for the sequential catalytic reaction to occur.

There is therefore a need in the art to provide a simplified bifunctional peroxidase-oxidase system, which will not compromise the enzymes stability, selectivity, and catalytic reaction.

The present invention addresses the above needs in the art by providing an enzymatic chimera capable of catalyzing in cascade the formation of hydrogen peroxide and (pseudo)halogenated compounds, thanks to its bifunctional myeloperoxidase-glucose oxidase activity.

Indeed, the present Inventors are herein the first to report the successful fusion of the open reading frames of a glucose oxidase (GOx from Penicillium amagasakiens) and of a myeloperoxidase (MPO from Rhodopirellula baltica or Homo sapiens). To do so, they genetically engineered a wide range of chimeric constructs, in which these enzymes were either fused directly end-to-end to each other, or via a short bridging amino acid sequence. Both enzymes expressed in a single open reading frame remained stable and catalytically active, even without a bridging sequence. Such result was rather unexpected given that these proteins are both very large constituents and that enzymes that derive catalytic activity from quaternary structure (which is the case for MPO and GO, each requiring dimerization to be active) can be easily deactivated by fusion with another enzyme (Ellis et al., ACS Catal. 2019, 9(12): 10812-10869). Even more surprising was the fact that the presence of the peptide bridge, referred herein as the peptide linker, improved the catalytic activity of the chimera, both at the level of the glucose oxidase activity and at the level of the myeloperoxidase activity. The chimera of the invention also proved to be microbicidal.

### SUMMARY OF THE INVENTION

In a first aspect, the invention relates to a non-naturally occurring polypeptide having a myeloperoxidase activity and a glucose oxidase activity.

In a preferred embodiment, the polypeptide of the invention is a fusion polypeptide comprising a myeloperoxidase coupled, preferably covalently coupled, to a glucose oxidase.

In a preferred embodiment, the C-terminus of the myeloperoxidase is coupled, preferably covalently coupled, to the N-terminus of the glucose oxidase.

In a preferred embodiment, the myeloperoxidase is coupled, preferably covalently coupled, to the glucose oxidase by a linker, preferably by a peptide linker.

In a preferred embodiment, the linker is a peptide linker comprising, or consisting of, the following amino acid sequence:

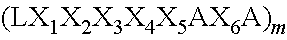

wherein
X₁ is glutamate or glycine,
X₂ is lysine or glycine,
X₃ is arginine or glycine,
X₄ is proline or vacant,
X₅ is glutamate or glycine,
X₆ is glutamate or glycine,
and m is an integer ranging from 1 to 2, preferably is 1;
   or is a substantially homologous peptide thereof, preferably deriving from said sequence by one or more conservative substitutions.

In a preferred embodiment, the peptide linker comprises, or consists of, any one of the following amino acid sequences: LEGGEAEA (SEQ ID NO: 4), LGKRGAGA (SEQ ID NO: 5), (LEKREAEA)₂ (SEQ ID NO: 6), LEKREAEA (SEQ ID NO: 7), or LEKRPEAEA (SEQ ID NO: 8); or is a substantially homologous peptide thereof, preferably deriving from any one of SEQ ID NO: 4 to 8 by one or more conservative substitutions.

In another preferred embodiment, the peptide linker comprises, or consists of, a polyglycine amino acid sequence, such as those comprising (G)m (m being an integer ranging from 2 to 10), in particular GGGGGGGG (SEQ ID NO: 9); or is a substantially homologous peptide thereof, preferably deriving from said sequence by one or more conservative substitutions.

In a preferred embodiment, the myeloperoxidase is a microbial myeloperoxidase, such as a myeloperoxidase from Rhodopirellula baltica, or is a mammalian myeloperoxidase, such as a mammalian myeloperoxidase from Homo sapiens.

In a preferred embodiment, the glucose oxidase is a microbial glucose oxidase, such as a glucose oxidase from Penicillium amagasakiens.

In a preferred embodiment, the polypeptide of the invention is in the form of a functional oligomer, or a mixture of functional oligomers.

Another aspect pertains to a nucleic acid encoding the polypeptide of the invention.

A further aspect relates to a vector comprising the nucleic acid of the invention.

An additional aspect is directed to a host cell comprising the vector of the invention.

Another aspect provides a method for obtaining the polypeptide of the invention, comprising at least the steps of: a) culturing in a medium a host cell of the invention, under conditions suitable for the expression of the polypeptide; and b) recovering said polypeptide.

A further aspect provides an antimicrobial composition, comprising the non-naturally occurring polypeptide of the invention.

In a preferred embodiment, the antimicrobial composition further comprises glucose or a source of glucose, and/or a halide or pseudohalide.

An additional aspect relates an in vitro use of the non-naturally occurring polypeptide or composition of the invention, for halogenating a non-halogenated organic compound.

Another aspect pertains to an in vitro or ex vivo use of the non-naturally occurring polypeptide or composition of the invention, for killing or inhibiting the growth of microorganisms.

A further aspect is directed to a non-naturally-occurring polypeptide or composition of the invention, for use as a medicament, preferably for the treatment of a microbial infection.

An additional aspect relates to (i) a non-naturally occurring polypeptide of the invention and (ii) glucose or a source of glucose and/or a halide or pseudohalide, as a combined preparation for simultaneous, separate or sequential use as a medicament, preferably for the treatment of a microbial infection.

Another aspect of the invention relates to a peptide linker comprising, or consisting of, the following amino acid sequence:

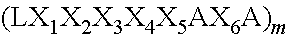

wherein
X₁ is glutamate or glycine,
X₂ is lysine or glycine,
X₃ is arginine or glycine,
X₄ is proline or vacant,
X₅ is glutamate or glycine,
X₆ is glutamate or glycine,
and m is an integer ranging from 1 to 2, preferably is 1;
   or is a substantially homologous peptide thereof, preferably deriving from said sequence by one or more conservative substitutions.

In a preferred embodiment, the peptide linker of the invention comprises, or consists of, any one of the following amino acid sequences: LEGGEAEA (SEQ ID NO: 4), LGKRGAGA (SEQ ID NO: 5), (LEKREAEA)₂ (SEQ ID NO: 6), LEKREAEA (SEQ ID NO: 7), or LEKRPEAEA (SEQ ID NO: 8); or is a substantially homologous peptide thereof, preferably deriving from any one of SEQ ID NO: 4 to 8 by one or more conservative substitutions.

### LEGENDS TO THE FIGURES

FIG. 1. Recapitulative scheme of the reaction catalyzed by the bifunctional MPO-GO polypeptide according to the invention.

FIG. 2. Catalytic efficiency of the RbMPO-GO chimeras according to the invention (coupled active sites). (A) Catalytic efficiency of coupled active sites towards glucose: all chimeras catalyze glucose in vitro, albeit with different efficiency depending on the nature of the peptide linker (if present) and oligomeric state (n≥1). (B) Catalytic efficiency of coupled active sites towards chloride: all chimeras catalyze NaCl in vitro, albeit with different efficiency depending on the nature of the peptide linker (if present) and oligomeric state (n≥1).

FIG. 3. Bactericidal activity and stability of the RbMPO-GO chimeras according to the invention. (A to N). A_{620 nm} measurement at 400 and 800 minutes in presence of different concentrations of the chimera (as shown on each Figure) and fixed concentrations of glucose (14 mM) along with SCN⁻ (25 mM) or Cl⁻ (80 mM), at different kinetics. For all experiments, kinetics were run during 16 hours at 37° C. Kinetic experiments were repeated after several days (d) of enzyme storage at 4° C. Data at 400 and 800 minutes have been derived from the kinetic curves. Bactericidal activity was measured at 37° C. and assessed in triplicates.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, nomenclatures used herein, and techniques of molecular biology, such as protein chimeric technology, or of enzymology, are those well-known and commonly used in the art.

The present invention may be understood more readily by reference to the following detailed description, included preferred embodiments of the invention, and examples included herein.

The present invention provides an artificial polypeptide capable of catalyzing, in a cascade reaction, the formation of hydrogen peroxide which in turn drives the formation of (pseudo)halogenated compounds.

To do so, the present invention relates, in a first aspect, to a non-naturally occurring polypeptide having a myeloperoxidase activity and a glucose oxidase activity.

As used herein, the terms “polypeptide” and “protein” are used interchangeably to refer to a precise succession of amino acids, also referred as amino acid sequence. As such, these terms include polypeptides of any size, preferably those of at least 50, 100, 250, 500, 600, 700, 800, 900, 1000, 1100, 1200, 1300, 1400, 1500 amino acids, and/or polypeptides that have undergone post-translational modifications.

A “non-naturally occurring” polypeptide refers herein to a polypeptide or protein that it is not found in nature (i.e. not a wild polypeptide). Such polypeptide can typically be a product of human agency, such as protein engineering.

The terms “activity”, “function”, “biological activity”, and “biological function” are equivalent and have to be understood as well known in the art. Preferably, such an activity is enzymatic. In the context of the present invention, the polypeptide is bifunctional, since it exhibits at least two functions or activities, which are herein a myeloperoxidase activity and a glucose oxidase activity.

A “myeloperoxidase activity” is typically characterized by the oxidation of halides or pseudo-halides into (pseudo)hypohalous acids, in the presence of hydrogen peroxide, according to the following reaction:

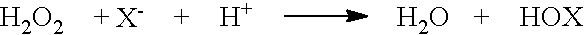

wherein X⁻ represents a halide or a pseudohalide.

The term “halide” refers to an ion of a halogen, and includes herein chloride (Cl⁻), bromide (Br⁻), or iodide (I⁻), and any combination thereof. The term “pseudohalide” refers to a polyatomic anion resembling the halides in their acid-base and redox chemistry, and includes herein thiocyanate (SCN⁻). Halides and pseudohalides are referred herein globally as (pseudo)halides.

A myeloperoxidase activity can be detected according to the protocols described in section 1.8 of Example 1 below, and/or measured according to the protocols described by Tenovuo et al. (Biochim Biophys Acta, 1986; 870(3): 377-84), Auer et al. (J Biol Chem., 2013; 288(38): 27181-27199) and/or Flemmig et al. (J Biol Chem., 2012; 287(33): 27913-23).

A myeloperoxidase activity can be typically be provided by a myeloperoxidase. A “myeloperoxidase” refers to an enzyme having a myeloperoxidase activity, especially when said enzyme is in the form of a haloenzyme (or holoenzyme), that is, when said enzyme is combined or complexed at least with its co-factor and optionally ions. Myeloperoxidases in the form of a haloenzyme are typically combined or complexed at least with heme and optionally calcium. Preferred myeloperoxidases according to the invention are as further described below.

A “glucose oxidase activity” is typically characterized by the production of hydrogen peroxide following oxidation of glucose or a source of glucose (such as dextrose or saccharose), according to the following reaction:

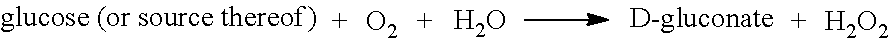

A glucose oxidase activity can be detected according to the protocols described in section 1.7 of Example 1 below, and/or measured according to the protocols described by Roth et al. (Proc Natl Acad Sci USA., 2003; 100(1):62-7), Courjean et al. (J Biotechnol., 2011; 151(1):122-9) and/or Ciaurriz et al. (J Colloid Interface Sci., 2014; 15; 414:73-81).

A glucose oxidase activity can be typically be provided by a glucose oxidase. A “glucose oxidase” refers to an enzyme having a glucose oxidase activity, especially when said enzyme is in the form of a haloenzyme (or holoenzyme), that is, when said enzyme is combined or complexed at least with its co-factor and optionally carbohydrate chains or glycans. Glucose oxidases in the form of a haloenzyme are typically combined or complexed at least with flavine adenine dinucleotide (FAD) and optionally glycosylated (such as in amino acid position 93 by reference to the numbering of the amino acid sequence SEQ ID NO: 12, the glycosylation being preferably GlucNac). Preferred glucose oxidases according to the invention are as further described below.

In a preferred embodiment, the polypeptide according to the invention is a fusion polypeptide comprising a myeloperoxidase coupled to a glucose oxidase.

As used herein, the term “fusion polypeptide” means a polypeptide created by joining two or more (poly)peptides together. To this end, the two or more (poly)peptides are coupled, either directly or indirectly, to one another. Such coupling can be performed by way of biological or physiochemical means. For example, for biological coupling, the fusion polypeptide can be a translation product of a chimeric gene construct that joins a first DNA sequence encoding a first (poly)peptide, with a second DNA sequence encoding a second (poly)peptide, so as to form a single open-reading frame.

Here, the myeloperoxidase and glucose oxidase of the fusion polypeptide according to the invention remained both catalytically active when the C-terminal extremity of the myeloperoxidase was coupled to the N-terminal extremity of the glucose oxidase.

Accordingly, in a preferred embodiment, the C-terminus of the myeloperoxidase is coupled to the N-terminus of the glucose oxidase.

In the context of the present invention, the coupling between these two enzymes is preferably a stable coupling for in vitro, ex vivo or even in vivo applications, typically by way of covalent coupling.

In a preferred embodiment, the myeloperoxidase, preferably the C-terminus of the myeloperoxidase, is covalently coupled to the glucose oxidase, preferably to the N-terminus of the glucose oxidase.

As used herein, the terms “covalently coupled”, “covalently bound”, “covalently linked”, “covalent coupling”, “covalent bonding” or “covalent linkage” refers to the interatomic linkage that results from the sharing of one or more pairs of electrons between two atoms, for example between two or more (poly)peptides. Typical examples of covalent bonds between two or more (poly)peptides include, without limitation, peptide bonds (covalent bonds that typically link amino acids to one another) and bridges (disulfide bridges which form between cysteine chains; or bridges between a lysine and a cysteine that are covalently linked by an oxygen atom). Two or more (poly)peptides may be covalently coupled, either directly or indirectly, to one another. An indirect coupling means that the two or more (poly)peptides are joined to one another through an intervening binding moiety or moieties, such as a linker. A direct coupling means that two or more (poly)peptides are joined to one another without any intervening binding moiety or moieties, such as a linker.

Against all expectations, the Inventors have demonstrated herein that the direct fusion of the myeloperoxidase to the glucose oxidase does not abolish the catalytic activity of each of said enzymes in the resulting polypeptide. Without being bound by theory, the Inventors believe that, upon such fusion, the individual enzymes retain their ability to fold independently of the remainder of the polypeptide chain.

Thus, in a preferred embodiment, the myeloperoxidase is covalently coupled, directly, to the glucose oxidase.

The Inventors have further demonstrated herein that the indirect fusion of the myeloperoxidase to the glucose oxidase, via a linker, can improve the catalytic activity of said enzymes in the resulting polypeptide—this improvement being by comparison to the direct fusion of the two enzymes. Without being bound by theory, the Inventors believe that, upon such fusion, the individual enzymes improve their ability to fold independently of the remainder of the polypeptide chain.

Accordingly, in a preferred embodiment the myeloperoxidase is covalently coupled to the glucose oxidase by a linker.

By “linker” or “spacer”, it is meant herein a chemical or biological moiety, synthetic or natural, capable of coupling two molecules to one another, and which may create a spatial separation between said molecules. Chemical linkers are well-known in the art and are typically made of polymer chains of varying lengths, which can be homo- or hetero-bifunctional with identical or non-identical reactive groups and comprise at least one atom, preferably at least one carbon atom. By contrast, biological linkers are typically made of nucleic acid(s) and/or amino acid sequence(s) of varying lengths, and comprise at least one nucleic acid and/or amino acid. Such linkers and methods of coupling molecules, in particular proteins, have been extensively described in the literature, notably by Chen et al. (Adv Drug Deliv Rev, 2013; 65(10):1357-1369), and in Thermo Scientific: Bionconjugation and crosslinking technical handbook (2018), and may thus be easily selected and designed by the skilled person in the art.

In the context of the present invention, biological linkers are particularly preferred, in particular peptide linkers.

By “peptide linker” or “peptidic linker”, it is intended to mean a biological linker as defined above and which is made amino acid sequence(s) of varying lengths. As a non-limiting indicative range, peptide linkers can be from about 2 amino acids to about 50 amino acids in length. Preferred peptide linkers according to the invention are those whose sequence comprises from about 3 to about 35 amino acids in length, preferably from about 3 to about 35 amino acids, more preferably from about 8 to about 18 amino acids. Peptide linkers are well-known in the art (Chen et al., Adv Drug Deliv Rev, 2013; 65(10):1357-1369, incorporated herein by reference in its entirety, notably Table 3) and can typically be qualified as rigid, semi-rigid, flexible or cleavable. Rigid peptide linkers exhibit relatively stiff structures and generally have a helical structure or are proline-rich. The amino acids proline, arginine, phenylalanine, glutamate and glutamine are typically found in rigid linkers. Proline may be also useful to create a hinge in the structure. Examples of rigid peptide linkers include, without limitation, the α-helix-forming peptide linkers such as A(EAAAK)mA (m being an integer ranging from 2 to 5) (SEQ ID NO: 1), the proline-alanine linker PAPAP (SEQ ID NO: 2), and the polyproline linker (P)m (m being an integer ranging from 2 to 8). Semi-rigid linkers are peptide linkers with limited flexibility, i.e. with a structure that is not stiff yet not fully flexible. Examples of semi-rigid linkers are described in WO2010080424A1 (incorporated herein by reference in its entirety). Flexible peptide linkers allow the coupled molecules to freely move relative to one another; such linkers are typically rich in small, non-polar or polar amino acids such as glycine and/or serine. Examples of flexible peptide linkers include, without limitation, polyglycine (G)m (m being an integer ranging from 2 to 10), and the glycine-serine linker (GGGGS)m (m being an integer ranging from 2 to 5) (SEQ ID NO: 3).

Rigid, semi-rigid and flexible linkers are stable in vivo, and thus do not allow the separation of coupled (poly)peptides. Cleavable peptide linkers, on the other hand, are susceptible to reductive or enzymatic cleavage; examples of such linkers include disulfide bridges or protease cleavage sites. The skilled person will readily understand that cleavable peptide linkers are not suited for the present invention. In a preferred embodiment, the linker used in the present invention is not a cleavable peptide linker. The peptide linker can accordingly be a rigid, a semi-rigid, or a flexible peptide linker.

The following peptide linkers are especially suited for the fusion polypeptide according to the invention.

In a preferred embodiment, the peptide linker comprises, or consists of, the following amino acid sequence:

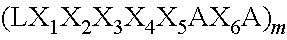

wherein
X₁ is glutamate or glycine,
X₂ is lysine or glycine,
X₃ is arginine or glycine,
X₄ is proline or vacant,
X₅ is glutamate or glycine,
X₆ is glutamate or glycine,
and m is an integer ranging from 1 to 2, preferably is 1;
   or is a substantially homologous peptide thereof, preferably deriving from said sequence by one or more conservative substitutions.

Two amino acid sequences are “homologous”, “substantially homologous” or “substantially similar” when one or more amino acids are replaced by one or more biologically similar amino acids, or when at least about 80% of the amino acids between the two sequences are identical, or at least about 90%, preferably at least about 95%, still preferably at least about 96% or 97%, more preferably at least about 98% or 99% are identical, yet the two sequences exhibit the same (or substantially the same) essential structure (e.g. tertiary, quaternary, rigid, semi-rigid, flexible, cleavable, etc.) and/or the same (or substantially the same) essential biological activity. In other words, two homologous amino acid sequences are said to be functional. By comparison to an amino acid sequence of reference, a homologous amino acid sequence can typically comprise e.g. silent mutations, conservative substitutions, or minor deletions of genetic material, which do not impact (or substantially impact) the structure or biological activity of the sequence of reference. Similar or homologous sequences can be identified by alignment using algorithms well-known in the art. For example, optimal alignment of sequences can be conducted by a global homology alignment algorithm, such as by the algorithm described by Needleman and Wunsch (Journal of Molecular Biology, 1970, 48(3): 443-53), or by computerized implementations of this algorithm. A global homology alignment may be preferred if the alignment is performed using sequences of the same or similar length. Percentage of identity can be preferably calculated over the entire length of the sequence of reference.

A “conservative substitution” as used herein denotes the replacement of an amino acid (or corresponding codon) by another, without altering the overall conformation and/or function of the (poly)peptide (or corresponding nucleic acid) of reference, including, but not limited to, replacement of an amino acid (or corresponding codon) with one having similar properties (such as, for example, polarity, hydrogen bonding potential, acidic, basic, shape, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Neutral hydrophilic amino acids, which can be substituted for one another, include asparagine, glutamine, serine and threonine.

By “substituted” or “modified”, the present invention includes those amino acids that have been altered or modified from naturally-occurring amino acids.

As such, it should be understood that in the context of the present invention, a conservative substitution is recognized in the art as a substitution of one amino acid for another amino acid that has similar properties. Examples of conservative substitutions are set out in the Table 1 below.

TABLE 1
Conservative substitutions I

| Side chain | |
|---|---|
| characteristic | Amino Acid |
| Non-polar | G, A, P, I, L, V |
| Polar uncharged | C, S, T, M, N, Q |
| Polar-charged | D, E, K, R |
| Aromatic | H, F, W, Y |
| Other | N, Q, D, E |

Alternatively, conservative amino acids can be grouped as described in Lehninger, 1975, as set out in Table 2 below.

TABLE 2
Conservative substitutions II

| | |
|---|---|
| Side chain | |
| characteristic | Amino Acid |

Non-polar (hydrophobic)

| | |
|---|---|
| Aliphatic | A, L, I, V, P |
| Aromatic | F, W |
| Sulfur-containing | M |
| Borderline | G |

Polar uncharged

| | |
|---|---|
| Hydroxyl | S, T, Y |
| Amides | N, Q |
| Sulfhydryl | C |
| Borderline | G |

Polar-charged

| | |
|---|---|
| Positively | K, R, H |
| charged (basic) | |
| Negatively | D, E |
| charged (acidic) | |

As a further alternative, exemplary conservative substitutions are set out in Table 3 below.

TABLE 3
Conservative substitutions III

| Original amino | |
|---|---|
| acid residue | Amino acid substitution |
| Ala (A) | Val (V), Leu (L), Ile (I) |
| Arg (R) | Lys (K), Gln (Q), Asn (N) |
| Asn (N) | Gln (Q), His (H), Lys(K), Arg (R) |
| Asp (D) | Glu (E) |
| Cys (C) | Ser (S) |
| Gln (Q) | Asn (N) |
| Glu (E) | Asp (D) |
| His (H) | Asn (N), Gln (Q), Lys (K), Arg (R) |
| Ile (I) | Leu (L), Val (V), Met (M), Ala (A), Phe (F) |
| Leu (L) | Ile (I), Val (V), Met (M), Ala (A), Phe (F) |
| Lys (K) | Arg (R), Gln (Q), Asn (N) |
| Met (M) | Leu (L), Phe (F), Ile (I) |
| Phe (F) | Leu (L), Val (V), Ile (I), Ala (A) |
| Pro (P) | Gly (G) |
| Ser (S) | Thr (S) |
| Thr (T) | Ser (S) |
| Trp (W) | Tyr (T) |
| Tyr (Y) | Trp (W), Phe (F), Thr (T), Ser (S) |
| Val (V) | Ile (I), Leu (L), Met (M), Phe (F), Ala (A) |

In a further preferred embodiment, the peptide linker of the present invention comprises, or consists of, any one of the following amino acid sequences: LEGGEAEA (SEQ ID NO: 4), LGKRGAGA (SEQ ID NO: 5), (LEKREAEA)₂ (SEQ ID NO: 6), LEKREAEA (SEQ ID NO: 7), or LEKRPEAEA (SEQ ID NO: 8); or is a substantially homologous peptide thereof, preferably deriving from any one of SEQ ID NO: 4 to 8 by one or more conservative substitutions.

Peptide linkers (LEKREAEA)₂ (SEQ ID NO: 6), LEKREAEA (SEQ ID NO: 7) and LEKRPEAEA (SEQ ID NO: 8) can essentially be characterized as rigid peptide linkers, while peptide linkers LEGGEAEA (SEQ ID NO: 4) and LGKRGAGA (SEQ ID NO: 5) can essentially be characterized as semi-rigid peptide linkers.

In another preferred embodiment, the peptide linker of the present invention comprises, or consists of, a polyglycine amino acid sequence, such as one comprising (G)m (m being an integer ranging from 2 to 10), in particular GGGGGGGG (SEQ ID NO: 9); or is a substantially homologous peptide thereof, preferably deriving from said sequence by one or more conservative substitutions.

Peptide linkers (G)m (m being an integer ranging from 2 to 10) and GGGGGGGG (SEQ ID NO: 9) can essentially be characterized as flexible peptide linkers.

In the context of the present invention, rigid and semi-rigid peptide linkers are especially preferred. Particularly preferred examples of such peptide linkers are: LEGGEAEA (SEQ ID NO: 4), LGKRGAGA (SEQ ID NO: 5), and (LEKREAEA)₂ (SEQ ID NO: 6).

In a preferred embodiment, the myeloperoxidase is a microbial myeloperoxidase, such as a myeloperoxidase from Rhodopirellula baltica, or is a mammalian myeloperoxidase, such as a mammalian myeloperoxidase from Homo sapiens.

Native myeloperoxidases and substantially homologous polypeptides thereof are encompassed herein.

For example, a particularly preferred myeloperoxidase according to the invention comprises, or consists of, the native amino acid sequence SEQ ID NO: 10 of Rhodopirellula baltica; or is a substantially homologous polypeptide thereof, preferably deriving from SEQ ID NO: 10 by one or more conservative substitutions.

As another example, a particularly preferred myeloperoxidase according to the invention comprises, or consists of, the native amino acid sequence SEQ ID NO: 11 of Homo sapiens; or is a substantially homologous polypeptide thereof, preferably deriving from SEQ ID NO: 11 by one or more conservative substitutions.

Conservative substitutions can especially be introduced into non-critical amino acids or in non-critical regions.

Amino acids that are critical for the biological activity of the preferred myeloperoxidases of the invention have actually been identified. These include amino acids at positions 199, 202, 203, 316, 317, and 407 by reference to the numbering of the amino acid sequence SEQ ID NO: 10, or amino acids at positions 257, 260, 261, 408, and 502 by reference to the numbering of the amino acid sequence SEQ ID NO: 11. These amino acids are more particularly Q199, D202, H203, E316, N317, and H407 in SEQ ID NO:13, or Q257, D260, H261, E408, M409 in SEQ ID NO: 11.

Accordingly, in a preferred embodiment, the myeloperoxidase comprises, or consists of, the native amino acid sequence SEQ ID NO: 10 of Rhodopirellula baltica; or is a substantially homologous polypeptide thereof, preferably deriving from SEQ ID NO: 10 by one or more conservative substitutions with the proviso that the following amino acids are conserved: Q199, D202, H203, E316, N317, and H407.

In another preferred embodiment, the myeloperoxidase comprises, or consists of, the native amino acid sequence SEQ ID NO: 11 of Homo sapiens; or is a substantially homologous polypeptide thereof, preferably deriving from SEQ ID NO: 11 by one or more conservative substitutions with the proviso that the following amino acids are conserved Q257, D260, H261, E408, M409.

In a preferred embodiment, the glucose oxidase is a microbial glucose oxidase, such as a glucose oxidase from Penicillium amagasakiens.

Native glucose oxidases and substantially homologous polypeptides thereof are encompassed herein.

For example, a particularly preferred glucose oxidase according to the invention comprises, or consisting of, the native amino acid sequence SEQ ID NO: 12 of Penicillium amagasakiens; or is a substantially homologous polypeptide thereof, preferably deriving from SEQ ID NO: 12 by one or more conservative substitutions.

As explained above, myeloperoxidases and glucose oxidases are each known in the art to require oligomerization to be catalytically active. This is why the Inventors have herein evaluated the different oligomeric states of the polypeptide according to the invention.

In a preferred embodiment, the polypeptide according to the invention is in the form of a functional oligomer (n≥1), or a mixture of functional oligomers (n≥1).

More precisely, the polypeptide according to the invention can be in the form of a functional monomer or a functional multimer, or a mixture thereof.

An “oligomer” or “oligomeric state” refers herein to the structural unit(s) that makes up an oligomeric polypeptide. The number (n) of these structural units, also known as the degree of oligomerization, can be equal or superior to 1 (n≥1). When n is superior to 1, the structural unit(s) are typically linked together either covalently or non-covalently. n is generally less than one hundred, usually less than thirty. An oligomer with n=1 is known as a monomer or a single unit, while an oligomer with n>1 can be referred as a multimer or a multi-unit. A monomer or single unit is typically made herein of one polypeptide (or polypeptide chain), while a multimer or a multi-unit is typically made of at least two polypeptides (or polypeptide chains). Oligomers of increasing length are called dimer (n=2), trimer (n=3), tetramer (n=4), pentamer (n=5), hexamer (n=6), heptamer (n=7), octamer (n=2), nonamer (n=9), decamer (n=10), etc.

While the different oligomers of the polypeptide according to the invention may exhibit different catalytic efficiencies, all remain essentially functional, in that they all exhibit a myeloperoxidase activity and a glucose oxidase activity. It is within the skill of the person in the art to select, if need be, the oligomers that have the desired level of myeloperoxidase and glucose oxidase activities. In order to reduce the time and cost of production, and increase production yield, one may also wish to favor the polypeptide of the invention in the form of a mixture of functional oligomers.

Methods for preparing the polypeptide of the invention are as described below.

The polypeptide according to the invention can be encoded by a nucleic acid.

By “nucleic acid” or “nucleotide sequence”, it is meant herein a precise succession of natural nucleotides (namely, A, T, G, C and U) or non-natural nucleotides. These terms encompass a single-stranded or double-stranded DNA, as well as the transcription product of said DNA, such as an RNA.

Accordingly, in a further aspect, the present invention pertains to a nucleic acid encoding the polypeptide as described herein.

Like the polypeptide it encodes, the nucleic acid is non-naturally occurring.

The nucleic acid of the invention can be prepared by methods well-known in the art, including, but not limited to, any synthetic and/or recombinant method. It is within the skill of the person in the art to design the nucleotide sequence of the nucleic acid so as to allow an efficient production of a functional polypeptide, such as by codon-optimization based on the desired expression system (e.g. host cell).

The nucleic acid according to the invention can advantageously be comprised in a vector in order to amplify this nucleic acid, or to express the polypeptide of the invention in a host cell.

It is thus a further aspect of the invention to provide a vector comprising the nucleic acid as disclosed herein.

Said vector can advantageously be comprised in a host cell, such as a prokaryotic or a eukaryotic cell. Accordingly, the vector can be a prokaryotic or eukaryotic vector.

The invention thus also relates to a host cell comprising the vector of the invention.

The term “vector” generally refers to a tool useful for performing procedures of molecular biology and genetic recombination. Such tool is commonly used and very well known in the art. This term encompasses vectors capable of replication in order to amplify a nucleic acid of interest (i.e. a cloning vector), or to express the polypeptide encoded by said nucleic acid in a host cell (i.e. an expression vector). These types of vectors are publicly available and include, without limitation, plasmids, cosmids, YACS, BACS, viral vectors (adenovirus, AAV, retrovirus such as lentivirus, EBV episome, etc.), and phage vectors. The vector is herein said to be recombinant in that it is not found in nature combined to the nucleic acid of the invention (i.e. it is not naturally-occurring).

Methods for inserting a nucleic acid into a vector are known to the skilled practitioner. Generally, a nucleic acid can be inserted into one or more restriction endonuclease site(s) using techniques well-known in the art (see, for example, the techniques described by Sambrook et al. in Molecular Cloning: A Laboratory Manual, 4^{th} edition, 2012). Nucleotide sequences allowing the transcription of said nucleic acid, the expression and/or purification of the protein encoded by said nucleic acid are preferably also contained in the vector. These sequences include, generally and without limitation, at least one sequence selected from one or more signal peptide sequence(s), an origin of replication, one or more gene(s) marker(s) selection, an enhancer element, a promoter, a transcription terminator, and possibly a sequence allowing purification of a protein. The insertion of such sequences in said vector can be done via standard ligation techniques known to those skilled in the art, such as mentioned above. It is additionally known to those skilled in the art that these nucleotide sequences can be selected based on the host cell in which the vector is intended to replicate, and/or in which the polypeptide encoded by the nucleic acid is intended to be expressed.

For example, depending on the selected replication origin, the vector may replicate in one or more host cells: the origin of replication of plasmid pBR322 is typically adapted to most Gram-negative bacteria, that of plasmid 2 is generally specific to yeast, and various origins of viral replication (SV40, polyoma, adenovirus, VSV or BPV) are particularly useful for cloning vectors in mammalian cells.

As another example, depending on the selected promoter, the nucleic acid may be transcribed and the corresponding polypeptide expressed in one or more host cells: promoters T7, Lac, trp, tac, λPL are typically specific for E. coli bacteria; promoters PHO5, GAP, TPI1, ADH are generally adapted to yeast; promoters of polyhedrin and P10 and their equivalents are conventionally used in insect cells; finally, promoter CMV, MT1, SV40, SRα, retroviral and gene promoters of a heat shock protein are particularly adapted to mammalian cells.

Non-exhaustive examples of selection marker genes typically contained in vectors are genes conferring resistance to an antibiotic or toxin (e.g., ampicillin, neomycin, zeocin, hygromycin, kanamycin, tetracycline, chloramphenicol, or combinations thereof), and genes allowing the compensation of an auxotrophic deficiency (e.g. the gene coding for dihydrolofate reductase DHFR allowing resistance to methotrexate, or still the TPI gene of S. pombe).

Non-exhaustive examples of nucleotide sequences that allow the purification of a polypeptide are the Histidine sequence (Histidine Tag or His×6), the FLAG sequence and the GST sequence. A cleavage sequence of a protease, such as VTE, may further be present in order to subsequently delete the purification sequence.

Non-exhaustive examples of prokaryotic vectors are: pET (Novagen), pQE70, pQE60, pQE-9 (Qiagen), pbs, pDIO, phagescript, psiX174, pbluescript SK, pbsks, pNH8A, pNH16A, pNH18A, pNH46A (Stratagene); ptrc99a, pKK223-3, pKK233-3, pDR540, pBR322, and pRIT5 (Pharmacia).

Non-exhaustive examples of eukaryotic vectors are: pWLNEO, pSV2CAT, pPICZ, pcDNA3.1 (+) Hyg (Invitrogen), pOG44, pXT1, pSG (Stratagene); pSVK3, pBPV, pCI-neo (Stratagene), pMSG, pSVL (Pharmacia); and pQE-30 (QLAexpress).

In a preferred embodiment, the vector of the invention is a prokaryotic vector, preferably the pET vector, such as the pET21a vector.

As used herein, the terms “host cell”, “cell” and “cell line” can be used interchangeably, and refer to a prokaryotic or eukaryotic cell in which the vector of the invention can be introduced, such as to amplify the nucleic acid as described above, and/or to express the polypeptide encoded by said nucleic acid. To this end, a host cell may be “transfected” or “transformed” by a process known in the art by which said vector is transferred or introduced into the host cell. Examples of such methods include, without limitation, electroporation, lipofection, calcium phosphate transfection, transfection using DEAE dextran, microinjection, and biolistics.

The choice of the host cell typically depends on the selected use, namely the cloning of the nucleic acid or the expression of the polypeptide encoded by said nucleic acid. The skilled person will be able to choose the appropriate host cell among the many cell lines that are publicly available, notably via the American Type Culture Collection (ATCC).

Examples of prokaryotic cells include, without limitation, bacteria such as Gram-negative bacteria of the genus Escherichia (e.g. E. coli BL21, C41, RR1, LE392, B, X1776, W3110, DH5 alpha, JM109, KC8), Serratia Pseudomonas, Erwinia Methylobacterium, Rhodobacter, Salmonella and Zymomonas, and Gram positive bacteria of the genus Corynebacterium, Brevibacterium, Bacillus, Arthrobacter, and Streptomyces.

Examples of eukaryotic cells include, without limitation, cells isolated from fungi, plants, and animals. Such cells notably include, without limitation, yeasts such as those of the genus Saccharomyces; cells from a fungus such as those of the genus Aspergillus, Neurospora, Fusarium or Trichoderma; animal cells such as HEK293 cells, NIH3T3, Jurkat, MEF, Vero, HeLa, CHO, W138, BHK, COS, COS-7, MDCK, C127, Saos, PC12, HKG; and insect cells such as Sf9, Sf21, Hi Five™ or of Bombyx mori.

In a preferred embodiment, the host cell of the invention is a prokaryotic cell, preferably of the genus Escherichia, more preferably E. coli such as E. coli BL21 (e.g. BL21 Star (DE3)).

The present Inventors have herein recombinantly produced the polypeptide of the invention, at a high yield, while still allowing a proper refolding of the polypeptide.

According to a further aspect, the invention relates to a method for obtaining the polypeptide of the invention, comprising at least the steps of:
a) culturing in a medium a host cell of the invention, under conditions suitable for the expression of the polypeptide; and
b) recovering the polypeptide.

The host cell used in said method is preferably as described above.

In a preferred embodiment, the host cell is a prokaryotic cell, preferably of the genus Escherichia, more preferably E. coli such as E. coli BL21 (e.g. BL21 Star (DE3)).

In step b), the polypeptide can be recovered from the host cell if said polypeptide is expressed intracellularly, and/or from the culture medium in which the host cell is cultured if said polypeptide is expressed extracellularly.

The skilled person in the art may use any conventional method allowing the recovery of said polypeptide. For example, if the polypeptide was expressed in a dissolved form in the host cell, the latter can be recovered by centrifugation and suspended in a buffer, then a cell-free extract can be obtained by destroying the cells through e.g. an ultrasonic homogenizer (sonication) or a cell disruptor optionally combined with an urea treatment.

In the context of the present invention, a reconstitution solution can be desired to ensure that the recovered polypeptide is fully folded and functional, i.e. is in a form of a haloenzyme. To do so, the polypeptide recovered in step b) can advantageously be solubilized in a solution comprising heme and FAD (or functional derivatives of heme or FAD) and optionally calcium. Heme and FAD are indeed the respective co-factors of myeloperoxidases and oxidases.

The polypeptide recovered in step b) can advantageously be purified, in a further step of said method, defined as step c). Preferably, said purification step allows the obtention of a 100%-purified or almost 100%-purified polypeptide.

The skilled person in the art may use any conventional method allowing the purification of said polypeptide. For example, if the polypeptide was recovered in a cell-free extract as detailed above, a purified sample can be obtained from the supernatant obtained by centrifugation of this extract, using a conventional method or combination of conventional methods to isolate and purify the polypeptide of the invention. These methods include, without limitation, solvent extraction, salting out with ammonium sulphate, desalting, precipitation with dialysis, filtration, ultrafiltration, organic solvent, preparative electrophoresis, isoelectric focusing, various chromatographic methods such as ion exchange chromatography (anionic, using for example a resin such as diethylaminoethyl (DEAE) Sepharose; or cationic, by using for example a resin such as S-Sepharose (Pharmacia), hydrophobic chromatography (using for example a resin such as butyl sepharose or phenyl sepharose), size-exclusion chromatography, affinity chromatography using antibodies, adsorption chromatography, chromatofocusing, high performance liquid chromatography (HPLC) and reversed phase HPLC, and any combinations thereof.

In the case where a chromatographic method is used in step c), one or more substeps can be performed and include, without limitation, the binding of the obtained polypeptide on a solid support, such as a chromatography column, a washing step, and an elution step. Said substeps can be repeated as many times as necessary in order to achieve the desired degree of purification of the polypeptide.

A size-exclusion chromatography column may be preferred to isolate the different oligomeric forms of the polypeptide. On the other hand, should the skilled practitioner wish to reduce the time and cost of production, one may use instead a desalting column to ensure the isolation of the polypeptide in the form of a mixture of all its functional oligomers.

The polypeptide recovered in step b) or purified in step c) may then be solubilized in a suitable buffer. A particularly preferred solubilization buffer according to the invention is buffer Tris pH7.5, preferably Tris pH7.5 CaCl₂.

Examples of a method allowing the obtention of the polypeptide of the invention is described in section 1.4 of Example 1 below, as well as in Eggenreich et al. (Biotechnology Reports, 2016, 10: 75-83). Such methods can notably allow the production of high yield of the polypeptide. As reported in the following Examples, the Inventors obtained from about 40 mg to about 600 mg of the polypeptide of the invention per liter of the cultured host cell.

Thanks to its capacity to catalyze in cascade the formation of hydrogen peroxide and (pseudo)halides into (pseudo)hypohalous acids—the latter being known to display potent bactericidal and antiviral activities —, the polypeptide of the invention can be used as an antimicrobial agent.

It is thus a further aspect of the invention to provide an antimicrobial composition, comprising the non-naturally occurring polypeptide as described herein.

The term “antimicrobial” refers herein to the killing or inhibition of the growth of a microorganism, and accordingly encompasses the terms “bactericidal”, “bacteriostatic”, “virucidal”, “virostatic”, “fungicidal”, “fungistatic”, “parasiticidal” and “parasitistatic”. Examples of microorganisms that can be killed or of which the growth can be inhibited according to the invention are further detailed below.

Additional components may be included, as desired. These components may be provided in a single composition, or may be separated into binary compositions for later mixing prior to use, as may be needed for a particular application. The skilled practitioner will indeed understand that one of these components may be left out and provided separately (hence, as a binary compositions) from the polypeptide so to preclude premature reaction and exhaustion of the components.

The skilled person will readily understand that the antimicrobial composition, whether single or binary, will preferably comprise a suitable substrate for the glucose oxidase such as glucose or a source of glucose (e.g. dextrose or saccharose), and/or a suitable substrate for the myeloperoxidase, such as a (pseudo)halide.

Thus, in a preferred embodiment, the antimicrobial composition, whether single or binary, further comprises glucose or a source of glucose, and/or a halide or pseudohalide.

The halide or pseudohalide may be selected from the group consisting of iodide, thiocyanate, bromide, chloride, and any combinations thereof, especially when the myeloperoxidase is a myeloperoxidase from Rhodopirellula baltica, or is from Homo sapiens. Particularly preferred (pseudo)halides according to the invention are thiocyanate and chloride, especially thiocyanate.

For the purpose of the invention, the antimicrobial composition may be in a form suitable for in vitro, ex vivo or in vivo use, depending on the localization of the microorganism to be targeted. The form of the composition will preferably be selected so as to obtain direct contact with the microorganism of interest. For example, should the antimicrobial composition be intended for administration to a subject, the composition may be in a form suitable for oral, nasal, topical, transdermal or parenteral administration, depending on the localization of the microorganism to be targeted.

The antimicrobial composition of the invention may additionally comprise a pharmaceutically acceptable excipient.

As used herein, the term a “pharmaceutically acceptable excipient” means an inactive or inert, and therefore nontoxic, compound of pharmaceutical grade but devoid of pharmacological action itself. Such excipient can be used to improve properties of a composition, such as shelf-life, retention time at the application site, consumer acceptance, etc. It includes, without limitation, surfactants (cationic, anionic, or neutral); surface stabilizers; other enhancers, such as preservatives, wetting or emulsifying agents; solvents; buffers; salt solutions; dispersion medium; isotonic and absorption delaying agents, and the like; that are physiologically compatible.

The skilled person may also wish to combine the antimicrobial composition of the invention with one or more therapeutic agents, either within the composition (single composition), or separately (binary composition). Such therapeutic agents include, for example, anti-bacterial agents, anti-viral agents, anti-fungicides, anti-parasitic agents, and any combinations thereof. For illustration purpose, examples of therapeutic agents suitable for the present invention include, without limitation, penicillins, cephalosporins, carbacephems, cephamycins, carbapenems, monobactams, aminoglycosides, glycopeptides, quinolones, tetracyclines, macrolides, fluoroquinolones, silver, copper, chlorhexidine, polyhexanide, biguanides, chitosan, and/or acetic acid.

The polypeptide or composition of the invention can be used in a broad range of industrial, pharmaceutical, medical, cosmetics and ecological applications, as well as in the food industry. It can notably be used for any purpose for which a free combination of a myeloperoxidase and a glucose oxidase have been reported.

For example, because of its myeloperoxidase activity, the polypeptide or composition of the invention can be useful for obtaining halogenated organic compounds of interest.

Thus, it is a further aspect of the invention to provide an in vitro use of the non-naturally occurring polypeptide or composition as described herein, for halogenating a non-halogenated organic compound.

In other words, the present invention relates to an in vitro method for halogenating a non-halogenated organic compound, said method comprising the step of contacting in vitro the polypeptide or composition as described herein with a non-halogenated organic compound.

As used herein, the terms “organic compounds” refers to gaseous, liquid, or solid chemical compounds whose molecules contain carbon.

An example of halogenation of non-halogenated organic compounds (RH) is as follows, using the polypeptide of the invention:

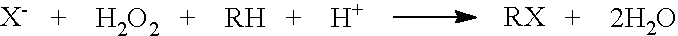

wherein RI represents a halogenated organic compound,
wherein H₂O₂ is provided by the oxidation of glucose (or source of glucose) by the glucose oxidase.

Particularly preferred halogenated organic compounds of interest include, without limitation, active organic compounds and chemical intermediates used during organic chemical synthesis, such as desinfectants, nutrients, pesticides, drugs, antibiotics, advantageously plant antibiotics, antioxydants, adhesives, and radiocontrast agents.

For illustration purpose, when the halide is iodide, iodinated compounds of interest can include, without limitation, phenolic compounds (e.g. mono-, di-, tri-, tetra-iodophloroglucinol, dibromoiodophenol and polymers thereof, as well as iodinated phlorotannins such as iodinated fuhalols, phlorethols, fucols, fucophlorethols, eckols and carmalols), volatile hydrocarbon compounds (e.g. iodoform, iodomethane, diiodomethane, bromoiodomethane, iodoethane, iodopropane, iodobutane, etc), terpenes, amino-acids derivatives (e.g. mono- and diiodotyrosine, which are thyroxine precursors) and fatty acids derivatives (e.g. eiseniaiodides). Iodomethane, diiodomethane, iodoform can be used as desinfectants or pesticides. Iodomethane, also known as methyl iodide, can additionally be used as a chemical intermediate during organic chemical synthesis, notably for methylating other compounds such as phenols, carboxylic acids, ammonia and derived amines, and for the industrial-scale production of acetic acid and acetic anhydride.

As another example, when the halide is iodide, radiocontrast agents obtainable by the invention can include, without limitation, 1,3,5-triiodobenzene and derivatives thereof, such as the ionic agents diatrizoate, metrizoate and ioxaglate, and the non-ionic agents ioversol, iopamidol, iohexol, ioxilan, iopromide and iodixanol. Such agents can be used for X-Ray imagery, such as fluoroscopy.

Because of its myeloperoxidase activity, the polypeptide or composition of the invention can also be useful for inhibiting the growth of a wide range of microorganisms, especially those that are pathological, such as those resistant to conventional therapies, in in vitro, ex vivo or in vivo applications.

Accordingly, it is thus a further aspect of the invention to provide an in vitro or ex vivo use of the non-naturally occurring polypeptide or composition as described herein, for killing or inhibiting the growth of microorganisms.

In other words, the present invention relates to an in vitro or ex vivo method for killing or inhibiting the growth of microorganisms, said method comprising the step of contacting in vitro or ex vivo the polypeptide or composition as described herein with a material or surface contaminated or at risk of being contaminated by microorganisms.

Such application can indeed be particularly suited to treat materials or surfaces that are contaminated or susceptible to be contaminated with microorganisms, so as to disinfect them, for example prior or after their use. The material or surface may be a surface of any device, laboratory material, surgery material, etc. such as a medical device, contact lenses and the like, especially those are intended to be used in contact with a subject (e.g. sutures, bandages, gauze, staples, zippers, etc.).

By “microorganisms”, it is meant herein bacteria, and also viruses, fungi, and parasites. For illustration purpose, examples of bacteria that can be efficiently inhibited by the present aspect of the invention include, without limitation, a wide range of Gram-negative or Gram-positive, such as Escherichia sp. (such as E. coli), Enterococcus sp., Staphylococcus sp., Streptococcus sp., Citrobacter sp., Enterobacter sp., Klebsiella sp., Proteus sp., Acinetobacter sp., Pseudomonas sp., Aeromonas sp., and Pasteurella sp., to name a few, but also Bacillus sp., Clostridium sp. and the like. Examples of fungi that can be efficiently inhibited by the present aspect of the invention include, without limitation, Aspergillus sp., Fusarium sp., Trichophyton sp., and the like. A particularly preferred microorganism according to the invention is a Escherichia sp., such as E. coli.

In addition, since the myeloperoxidase in the polypeptide operates by an entire different mechanism of action than molecules involved in traditional therapies such as antibiotics, in some instances, the present aspect of the invention may be useful to eliminate drug-resistant, multi-drug resistant, or antibiotics-resistant microorganisms. For illustration purpose, examples of drug-resistant microorganisms include, without limitation, the pathogenic bacteria MRSA (methicillin-resistant Staphylococcus aureus), VRSA (Vancomycin-resistant Staphylococcus aureus), VRE (Vancomycin-Resistant Enterococcus), Penicillin-Resistant Enterococcus, PRSP (Penicillin-resistant Streptococcus pneumoniae), the isoniazid/rifampin-resistant Mycobacterium tuberculosis, and other antibiotic-resistant strains of E. coli, Salmonella, Campylobacter, and Streptococci.

In another aspect, the present invention relates to a non-naturally-occurring polypeptide or composition as described herein, for use as a medicament, preferably for the treatment of a microbial infection.

In particular, the present invention is directed to the use of the polypeptide or composition as described herein, for the preparation of a medicament, preferably for the treatment of a microbial infection.

The present invention also provides a method for treating a microbial infection in a subject in need thereof, said method comprising the administration of a therapeutically effective amount of the polypeptide or composition as described herein, to said subject.

The present invention further relates to (i) a non-naturally occurring polypeptide of the invention and (ii) glucose or a source of glucose and/or a halide or pseudohalide, as described above, as a combined preparation for simultaneous, separate or sequential use as a medicament, preferably for the treatment of a microbial infection.

Generally speaking, the term “treatment” or “treating” means obtaining a desired physiological or pharmacological effect depending on the degree of severity of the symptom or disorder of interest, or risks thereof, i.e. herein, depending on the degree of severity of the microbial infection, or risks of developing such symptom or disorder. The effect may be prophylactic in terms of a partial or complete prevention of the symptom or disorder and/or may be therapeutic in terms of a partial or complete cure of the symptom or disorder. The term “prophylactic” characterizes the capacity to avoid, or minimize the onset or development of a symptom or disorder before its onset (for example, after exposure the microorganism, but before the onset of associated symptom). The term “therapeutic” refers to the capacity to inhibit the symptom or disorder (i.e. arresting the development thereof), and/or to relieve said symptom or disorder (i.e. regression leading to an improvement). In the context of the invention, a prophylactic effect is generally said to be achieved when e.g. an asymptomatic subject exposed to a microorganism remains asymptomatic or quasi-asymptomatic after treatment according the invention (for example, no development of an infection), while a therapeutic effect is typically said to be achieved when e.g. a symptomatic subject infected with a microorganism recovers after treatment according to the invention (for example, partial or complete relief of the infection).

Due to its wide spectrum of activity, the polypeptide or composition of the invention may be advantageously used in the treatment of polymicrobial infections. Polymicrobial diseases involve multiple infectious agents and can include complex, complicated, mixed, dual, secondary, synergistic, concurrent, polymicrobial, or coinfections. Polymicrobial diseases include, for example, infections associated with abscesses, AIDS-related opportunistic infections, conjunctivitis, gastroenteritis, hepatitis, multiple sclerosis, otitis media, periodontal diseases, respiratory diseases, and genital infections.

The polypeptide or composition of the invention may also be advantageously used in the treatment of microbial infections that are resistant to traditional therapies. Examples of such infections include those linked to microorganisms that are drug-resistant as described above.

As indicated above, the treatment according to the invention can be achieved by administering a therapeutically effective amount of the polypeptide or composition as described herein, to a subject in need thereof, using any suitable scheme of administration. For instance, said administration can be performed orally, nasally, topically, transdermally, parenterally, or any combinations thereof, depending on the type of infection affecting the subject. The route of administration will preferably be designed to obtain direct contact of the polypeptide or composition with the infecting microorganism. The dose and/or scheme of administration can be easily determined and adapted by the skilled practitioner, in accordance with the age, weight and/or severity of the infection from which the subject suffers.

It shall be further understood that the “subject” to be treated according to the invention is preferably a human or animal, more preferably a human.

In a further aspect, the invention relates to a peptide linker comprising, or consisting of, the following amino acid sequence:

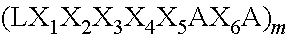

wherein
X₁ is glutamate or glycine,
X₂ is lysine or glycine,
X₃ is arginine or glycine,
X₄ is proline or vacant,
X₅ is glutamate or glycine,
X₆ is glutamate or glycine,
and m is an integer ranging from 1 to 2, preferably is 1;
   or is a substantially homologous peptide thereof, preferably deriving from said sequence by one or more conservative substitutions.

In a preferred embodiment, the peptide linker of the present invention comprises, or consists of, any one of the following amino acid sequences: LEGGEAEA (SEQ ID NO: 4), LGKRGAGA (SEQ ID NO: 5), (LEKREAEA)₂ (SEQ ID NO: 6), LEKREAEA (SEQ ID NO: 7), or LEKRPEAEA (SEQ ID NO: 8); or is a substantially homologous peptide thereof, preferably deriving from any one of SEQ ID NO: 4 to 8 by one or more conservative substitutions. More particularly preferred peptide linkers of the invention are: LEGGEAEA (SEQ ID NO: 4), LGKRGAGA (SEQ ID NO: 5), and (LEKREAEA)₂ (SEQ ID NO: 6).

Conservative substitutions are as describe above.

The above-described peptide linkers can be used in vitro to couple molecules to one another, preferably by covalent coupling. Examples of such molecules are a myeloperoxidase and a glucose oxidase as described herein.

The present invention will be better understood in the light of the following detailed experiments. Nevertheless, the skilled artisan will appreciate that the present examples are not limitative and that various modifications, substitutions, omissions, and changes may be made without departing from the scope of the invention.

### EXAMPLES

The aim of this study was to produce an artificial bifunctional enzyme, which is capable to catalyze the formation of (pseudo) hypohalogenated compounds from glucose and (pseudo) halides. To this end, the Inventors selected the open reading frame of the myeloperoxidase from Rhodopirellula baltica (EXAMPLE 1) or from Homo sapiens (EXAMPLE 2) fused to the glucose oxidase from Penicillium amagasakiens. Two types of chimeras were obtained by genetic engineering: one with an original peptide linker, and one without such linker between the two enzymes. The results demonstrate that the presence of this original linker is key to facilitate the activation of glucose oxidase inside the chimera and enable the formation of sufficient microbicidal compounds to kill an Escherichia coli strain used for antimicrobial resistance analysis. To understand the key properties of this original linker, several mutations were performed: the sequence and length of the linker appeared to be important for the topology, oligomerization, kinetics as well as microbicidal properties of the chimera.

### Example 1

### 1. Materials and Methods

### 1.1. Construction of the Chimera RbMPO-GOx Devoid of Peptide Linker

PCR amplification of the GOx_{penag} ORF from the pPiczα-GOx_{penag} vector (Zhang, Biotechnology Advances, 2011; 29(6): 715-725) was performed so as to introduce two restriction sites, i.e. NdeI and XhoI. The amplified DNA was then digested at these two sites and the digested fragment containing the GOx_{penag} ORF was ligated into a pET21a plasmid (digested with NdeI and XhoI).

The pEt21a-RbMPO-6His vector was digested with XhoI and the pET21aGOx_{penag} with NdeI. The two linearized vectors were used as megaprimers using the two following external primers: RbMPO C-Ter and GOx_{penag} (Table 4). The PCR product was subsequently digested with DpnI for 2 h at 37° C. and purified. A final ligation step preceded the transformation of the new construct into DH5α. To check whether the chimeric construct was correct, the clones were assessed in colony PCR using two primers that amplify the fusion zone: GOx65rc and RbMPOH407A (Table 4).

The pET21a-RbMPO-GOx plasmid was obtained.

TABLE 4
Primers

| Primers | Nucleotide sequence | SEQ ID |
|---|---|---|
| | (from 5′ to 3′) | NO: |
| RbMPO C-Ter | agcgaggacgataaaatcc | SEQ ID |
| | aggccgcgaattctttccg | NO: 13 |
| | c | |
| GOX_{penag} N-Ter | tacctgcctgcccaacaga | SEQ ID |
| | ttgatgtccagtctag | NO: 14 |
| GOx65rc | ggtgccaaagatttctcca | SEQ ID |
| | taagcatttg | NO: 15 |
| RbMPOH407A | ccgccgcgtttcggttggg | SEQ ID |
| | ggcgagcacgcttcgtg | NO: 16 |

1.2. Construction of the Chimera RbMPO-GOx with a Peptide Linker

The pPiczαGOx_{penag} vector was digested by the restriction enzyme XhoI that was present at two locations on either side of the enzyme ORF.

The pET21aRbMPO-6His plasmid was digested with XhoI, which was located at the C-Ter of the enzyme just before the 6His tag. The linearized plasmid underwent ligation with the ORF of GOx_{penag} in order to obtain the final chimeric construction. In this construction, a nucleic acid sequence coding for the peptide linker LEKREAEA (SEQ ID NO: 7) is present directly in 5′ of the ORF of RbMPO. The construction was verified after transformation in DH5a bacteria by performing a colony PCR with the oligonucleotides GOx 65rc and RbMPOH407A.

The pET21a-RbMPO-LEKREAEA-GOx plasmid was obtained.

1.3. Construction of the Chimera RbMPO-GOx with Alternative Peptide Linkers

A Quick-Change kit from Stratagene or a Q5 site directed mutagenesis kit from NEB Biolabs was used to modified the chimeras, using the primers of Table 5.

The pET21a-RbMPO-GOx vector (no linker) was used as matrix to add a sequence coding 8 Gly (SEQ ID NO: 9) or LGKRGAGA (SEQ ID NO: 5).

The pET21a-RbMPO-LEKREAEA-GOx vector was used as matrix to replace the linker with the sequence i) LEKREAEALEKREAEA (SEQ ID NO: 6), ii) LEGGEAEA (SEQ ID NO: 4), or iii) LEKRPEAEA (SEQ ID NO: 8).

The five following plasmids were accordingly obtained with a modified linker, namely:

pET21a-RbMPO-GGGGGGGG-GOx,
pET21a-RbMPO-LGKRGAGA-GOx,
pET21a-RbMPO-LEKREAEALEKREAEA-GOx,
pET21a-RbMPO-LEGGEAEA-GOx,
and
pET21a-RbMPO-LEKRPEAEA-GOx.

TABLE 5
Primers

| | | |
|---|---|---|
| | Nucleotide sequence | |
| Primers | (from 5′ to 3′) | SEQ ID NO: |

peptide linker: GGGGGGGG (SEQ ID NO: 9)

| | | |
|---|---|---|
| Forward primer | ggcggcggcggctacctgcctgcccaacagattgatg | SEQ ID NO: 17 |
| Reverse primer | gccgccgccgccagcgaggacgataaaatccaggccgc | SEQ ID NO: 18 |

peptide linker: LGKRGAGA (SEQ ID NO: 5)

| | | |
|---|---|---|
| Forward primer | ggcgccggcgcctacctgcctgcccaacagattgatg | SEQ ID NO: 19 |
| Reverse primer | tcttttgccgagagcgaggacgataaaatccaggccgc | SEQ ID NO: 20 |

peptide linker: LEKREAEALEKREAEA (SEQ ID NO: 6)

| | | |
|---|---|---|
| Forward primer | gagggctgaagcttacctgcctgcccaacagattgatg | SEQ ID NO: 21 |
| Reverse primer | tcttttctcgagagcttcagcctctcttttctcgagagcgagg | SEQ ID NO: 22 |

peptide linker: LEGGEAEA (SEQ ID NO: 4)

| | | |
|---|---|---|
| Forward primer | cgtcctcgctctcgagggcggcgaggctgaagcttacc | SEQ ID NO: 23 |
| Reverse primer | ggtaagcttcagcctcgccgccctcgagagcgaggacg | SEQ ID NO: 24 |

peptide linker: LEKRPEAEA (SEQ ID NO: 8)

| | | |
|---|---|---|
| Forward primer | cgtcctcgctctcgagaaaagaccggaggctgaagcttcctgaa | SEQ ID NO: 25 |
| Reverse primer | ggcaggtaagcttcagcctccggtcttttctcgagagcgaggacg | SEQ ID NO: 26 |

### 1.4. Production and Purification of the Chimeras

Each pET21a vector expressing a chimera was transformed in E. coli BL21Star (DE3) and bacterial clones were grown on LB agar ampicilin and chloramphenicol nutrient medium. 1 L was incubated with 10 mL of a pre-culture performed the previous day and OD_{600 nm} was measured until exponential phase of bacterial growth (0.5-0.8). An induction at 500 μM IPTG was used to trigger transcription and translation of the chimera. After 24 hours at 22° C. the cultures were pelleted. The bacteria were crushed at 2200 bar (4° C.) and the bacterial pellet was washed with a solution of 2 M urea, 50 mM Tris, 5.5 mM CaCl₂ pH 7.5. The chimera, which has a size of 142088.88 Da, was produced in the inclusion bodies of the bacteria so treatment of the pellet with a solution of 8 M urea, 50 mM Tris, 5.5 mM CaCl₂ pH 7.5 during 4 hours at 4° C. was required to resolubilise all the proteins. The supernatant recovered after centrifugation was dripped into a reconstitution solution (Tris 20 mM pH 7.5, 5.5 mM CaCl₂, 5 μM hemin, 200 μM FAD, 1 mM oxidised glutathione, 1 mM reduced glutathione and 10% v/v glycerol) for 5, 8 or 15 days. The mixture was concentrated first with a concentration cassette (Sartorius PES Cassettes Vivaflow 200 with a cut off of 30 kDa) and then with a 30 kDa amicon (Merck-Millipore amicon Ultra-15, PLHK, membrane Ultracel-PL) to obtain a volume lower than 13 mL (first set of experiments) or 7.5 mL (second set of experiments).

For the first set of experiments, the resulting protein solution was injected onto a size exclusion chromatography (Hiload 26/600 200pn Cytiva: fractionation range 10000-600000 Da). After isocratic elution (50 mM Tris, 5.5 mM CaCl₂ pH 7.5), the peak(s) containing the chimera was concentrated. The column was calibrated with gel filtration standard from Biorad. This step allowed the separation of the different oligomeric states (n≥1) of each chimera, for further characterization of each oligomeric state.

For the second set of experiments, the resulting protein solution was desalted on a PD10 column equilibrated with Tris 50 mM pH 7.5, then lyophilized and stored in the lyophilized form at 4° C. This allowed the isolation, in a single mixture, of all the different oligomeric states of a chimera, for further characterization of said mixture.

### 1.5. UV Spectrum

The UV spectrum of each chimera was assessed using a spectrophotometer, which scans the protein sample (diluted 100-fold) from 800 to 200 nm. The theoretical epsilon of 144285 M1·cm-1 and Beer Lambert's law allowed the determinization of the enzyme concentration after purification.

### 1.6. SDS-PAGE

Mini-PROTEAN® Precast Gels (Bio-rad) were used. These gels were characterised by a gradient of 4-15% acrylamide. Protein samples to be analysed were homogenised with Filler Blue 4X (containing SDS and β-mercaptoethanol) and then heated to 95° C. for 5 minutes. Denaturing migration buffer (SDS) and 35 mM amperage per gel were used to migrate proteins according to their molecular weight. After migration, the gel was stained for 3 hours in a Coomassie blue solution and then undergoes successive fading steps in a 10% acetic acid 30% ethanol solution.

### 1.7. Glucose Oxidase Activity of the Chimeras

The D-Gluconic acid/D-Glucono-δ-lactone kit from Megazyme was used to assess the glucose oxidase activity of the chimeras. The day before, a solution containing 5 μM of chimera, 250 mM glucose and 50 mM NaPi pH6 was incubated at 37° C. The next day, 15.7 μL of this solution was taken and the activity of the GOx_{penag} in the chimera was measured at 340 nm, which corresponded to the wavelength of the final product of the kit, i.e. NADPH. The concentration of D-gluconic acid produced by the chimera after one night of reaction was calculated with the following relationship:

$$[ gluconate ] ( g / L ) = \frac{{V}_{reaction} * {M}_{weight}}{{ε}_{g l uconate} * {V}_{sample}} Δ {A}_{340 n m}$$

where ε_{gluconate}=6300 M⁻¹·cm⁻¹ and molecular weight of the same compound was 196.1 g/mol.

### 1.8. Myeloperoxidase Activity of the Chimeras

### Using Chloride as a Substrate

The fluorescent probe aminophenyl fluorescein (APF) (λ_{excitation}=485 nm and λₑₘᵢₛₛᵢₒₙ=525 nm) was used to measure the HOCl production by the chimera, as a measure of its chlorination activity. Three tests were performed: (1) at different glucose concentrations (0, 1, 2, 3, 4, 5, 6, 8, 10, 15, 20 mM) with fixed concentrations of chimera (1 μM) and NaCl (20 mM); (2) at different NaCl concentrations (0, 10, 20, 50, 100, 250, 500, 750 μM and 1 mM) with fixed concentrations of chimera (1 μM) and glucose (20 mM); (3) at different H₂O₂ concentrations (0, 0.01, 0.02, 0.05, 0.079, 0.1, 0.25, 0.5, 0.75, 1, 2, 4, 6, 8, 10, 20, 40, 60, 80, 100, 200, 500 mM) with fixed concentrations of chimera (1 μM) and NaCl (20 mM). For all experiments, 10 μM APF was used in each assay with 50 mM NaPi buffer pH7.5 QSP 20 μL (384-well plate). Each experiment was performed in triplicate. A 10-30 min. kinetic was performed for each range value and the increase rate of fluorescence intensity was compared to the standard fluorescein range. The measured fluorescence per second was thus proportional to the amount of fluorescein, which in turn was proportional to the amount of HOCl produced by the chimeras.

Steady-state kinetic parameters were determined by adjustment of experimental data to the following equations depending on the appearance of saturation curves:

$${k}_{s s} = \frac{{k}_{c a t} * [ S ]}{{K}_{M} + [ S ]} \qquad Eq . 1$$

$${k}_{s s} = \frac{{k}_{c a t} * [ S ]}{{K}_{M} + [ S ] * ( 1 + \frac{[ S ]}{{K}_{i}} )} \qquad Eq . 2$$

$${k}_{s s} = {k}_{2} * [ S ] + b \qquad Eq . 3$$

where kss corresponds to the steady-state rate constant (initial velocity divided by chimera concentration);
kcat is the catalytic constant (maximal velocity divided by chimera concentration); [S] the concentration of substrate (glucose or NaCl);
K_{M} the Michaelis constant corresponding to the concentration of substrate (glucose or NaCl) where 50% of the maximal velocity was observed;
Ki the inhibition constant when inhibition per excess of substrate (glucose or NaCl) where observed; and
k₂ the slope when no saturation was observed in the range of concentration of substrate tested.

### Using Thiocyanate as a Substrate

Initial rate measurements under steady state conditions were made with a BioLogic SFM400 stopped-flow by using a single-mixing mode and Xe/Hg lamp and a TC100/10 cell. One syringe was filled with the chimera and another one with glucose and NaSCN. The concentrations of the chimera and glucose were fixed (300 nM and 50 mM, respectively), and measurement was carried out at 37° C. and at 240 nm. The appearance of −OSCN (hypothiocyanate) was measured at 240 nm at 37° C. in a 50 mM pH6 sodium phosphate buffer. At least five shots of 120 s were performed for each value of the NaSCN substrate range with the following sample period: 20 μs, 20 ms, 20 ms with 2000 points per period. Slopes were calculated from shots average for each concentration and kₛₛ (s⁻¹) were determined using slopes with the following formula:

$${k}_{s s} = \frac{slope ( Abs \cdot {s}^{- 1} )}{{ε}_{OSCN} ( {M}^{- 1} \cdot {cm}^{- 1} )} \frac{1}{[ RbMPO ] ( M )} \frac{1}{l ( 1 cm )}$$

The molar extinction coefficient of −OSCN is ε_{-OSCN}=951 M⁻¹·s⁻¹. Collecting data were analysed with Origin software and kₛₛ values were fitted with Michaëlis-Menten equation.

### 1.9. Microbicidal Activity and Stability of the Chimeras

Glycerol stock of the E. coli ATCC 25922 strain—the strain recommended for conducting antibiograms/for assessment of antimicrobial activity of compounds of interest—was deposited on a Petri dish containing Tryptic Soy Broth (TSB) agar medium for 16 hours at 37° C. 10 mL of TSB medium was inoculated with an isolated bacterial colony and the pre-culture was agitated at 190 rpm and 37° C. overnight. 25 mL of TSB medium were inoculated with the volume of pre-culture necessary to obtain an OD620 nm of 0.09. When the latter reached the exponential phase, a bacterial dilution of 10 mL (in TSB) was performed to obtain a bacterial quantity of 2·10⁶ CFU/mL with the correspondence of 1 OD620 nm to 1·10⁸ CFU/mL. The microbicidal tests were performed in 96-well microplates (Greiner Bio-One™, Cellstar™ μclear™, white, flat-bottom) in a final volume of 100 μL per well, with each experimental condition in triplicate. In each well, 50 μL of bacteria were plated to be at 1·10⁶ CFU/mL in all conditions. OD620 nm was measured with a Wallac Victor2 1420 microplate reader or a SpectraMax® Paradigm® (Molecular Devices), every 15 min for 16 h with shaking before each reading as well as a thermostatic atmosphere at 37° C. Those experiments were repeated after several days of enzyme storage at 4° C. under the same conditions, thereby also assessing the stability of the chimera.

### 2. Results

### 2.1. Construction of the RbMPO-GOx Chimeras

After bacterial transformation step, several clones were selected and tested by colony PCR to verify the presence the chimeric construct, using the primers GOx65rc and RbMPOH407A for the chimera without a peptide linker, or the primers described in above Table 5 for chimeras comprising a peptide linker. Sequencing confirmed the presence of the complete chimeric sequence for each construct.

### 2.2. Production and Purification of the RbMPO-GOx Chimeras

In the first set of experiments, all chimeras were purified after 8 days of reconstitution with heme and FAD, which are co-factors of RbMPO and GOx respectively. For each chimera, about 40 mg of the protein were obtained per liter of bacterial culture. Depending of the linker peptide sequence, one or more peaks were detected during the purification process. Since a size exclusion chromatography was performed, the first peak corresponded to molecules with a higher molecular weight than those contained in the following peaks. Free FAD molecules and the other components of the reconstitution solution were eluted in the total volume of the S200 column. Calibration with standard proteins allowed the determination of the oligomeric states (n≥1) of the chimeras based on their elution volume used to calculate the partition coefficient. In total, 7 chimeras, with or without a peptide linker, were purified and depending on their elution volume and purity, different oligomeric states of these chimeras were analysed and studied. These oligomeric states are summarized in Table 6 below. In parallel, SDS-PAGE analysis showed that the different peaks corresponded to pure chimera with a band with a molecular weight of about 150 kDa

UV spectrum of each chimera, including the spectrum of free heme, were obtained (data not shown). The spectrum of free heme (RbMPO cofactor) displays two main peaks, one at 385 nm and the other at 615 nm. For the chimeras, the fixation of heme in the active site of RbMPO can be evidenced by a shift of these two peaks, respectively at 412 nm and 637 nm. A peak at 280 nm is characteristic of tyrosine and tryptophan amino acids, which are components of RbMPO and GOx.

The following information could be deduced from these spectra:

$${R}_{z} ( \frac{{A}_{412 n m}}{{A}_{280 n m}} ) , ratio ( \frac{{A}_{280 n m}}{{A}_{260 n m}} )$$

as well as the mass percentage of each chimera in each oligomeric state. Rz is indicative of the presence of heme in the chimera. The ratio

$$( \frac{{A}_{280 n m}}{{A}_{260 n m}} )$$

is indicative of the purity of the chimera towards DNA and/or can indicate the presence of FAD in the active site of GOx. These parameters are also summarized in Table 6.

Apparent molecular weight (MWapp) of chimeras was determined from their elution volume (and then their partition coefficient K_{AV}) and calibration curve of S200 column. Their apparent oligomeric state was then obtained by dividing MWₐₚₚ by MW of the monomer (MW_{Monomer}). Relative quantity obtained for each oligomeric state of each chimera was determined from the concentration of each chimera then from UV visible spectra using Beer-Lamber, dilution factor of the chimera and molecular extinction coefficient. See also Table 6.

TABLE 6
Apparent oligomeric states (n ≥ 1) and spectral properties of the chimeras. “Out of
range” means that the proteins were eluted in the dead volume of the S200 column; “n.a” means
not applicable; “excluded” means that the molecular weight is higher than 600000 Da above
the S200 column splitting domain.

| RbMPO-GOx chimera | MWₐₚₚ (Da) | $\frac{M {W}_{a p p}}{M {W}_{m o n o m e r}}$ | Oligomeric state (n ≥ 1) | Rz | $\frac{{A}_{280}}{{A}_{260}}$ | % |
|---|---|---|---|---|---|---|
| No linker | out of range | n.a. | multimer | 0 | 0.82 | 34.8 |
| | 177688 | 1.3 | monomer | 0.29 | 1.21 | 65.2 |
| LEKREAEA | 421887 | 3 | trimer | 0.45 | 1.18 | 100 |
| LEKRPEAEA | out of range | n.a. | multimer | 0.19 | 0.78 | 27.6 |
| | 426266 | 3 | trimer | 0.28 | 1.16 | 42.9 |
| | 230876 | 1.6 | dimer/monomer | 0.32 | 1.34 | 29.5 |
| LEKREAEALEKREAEA | out of range | n.a. | excluded | 0.40 | 0.85 | 62 |
| | 393908 | 2.77 | trimer | 0.43 | 1.28 | 19 |
| | 206912 | 1.46 | dimer/monomer | 0.48 | 1.28 | 19 |
| LEGGEAEA | out of range | n.a. | multimer | 0 | 0.4 | 1.2 |
| | 224475 | 1.6 | dimer/monomer | 0.34 | 0.87 | 14.3 |
| | 162281 | 1.1 | monomer 1 | 0.42 | 1 | 69.9 |
| | 112920 | 0.5 | monomer 2 | 0.49 | 1.21 | 14.6 |
| LGKRGAGA | out of range | n.a. | multimer | 0.35 | 0.81 | 48.1 |
| | 404412 | 2.8 | trimer | 0.24 | 0.66 | 20.4 |
| | 203363 | 1.4 | dimer/monomer | 0.35 | 0.77 | 31.5 |
| GGGGGGGG | out of range | n.a. | multimer | 0.23 | 0.71 | 30.2 |
| | 444645 | 3.1 | trimer | 0.28 | 0.79 | 22.6 |
| | 213812 | 1.5 | dimer/monomer | 0.37 | 0.98 | 35.3 |
| | 112920 | 0.8 | monomer | 0.31 | 0.89 | 11.9 |

The most active chimera appeared to be RbMPO-LEGGEAEA-GOx due to its monomeric state, the best Rz, and highest ratio

$$( \frac{{A}_{280 n m}}{{A}_{260 n m}} )$$

compared to the other chimeras.

In the second set of experiments, the chimera RbMPO-LEGGEAEA-GOx was produced and purified as a mixture (i.e. no separation of oligomeric states). About 151 mg of protein were obtained from 250 ml of bacterial culture, which is a considerable production yield compared to the first set of experiments that were conducted with individual oligomeric states of the chimera.

### 2.3. Enzyme Characterization of Individual Active Sites of the Chimeras

Two enzymatic tests were undertaken to determine the activity of the two active sites independently of each other.

### 2.3.1. Glucose Oxidase Activity

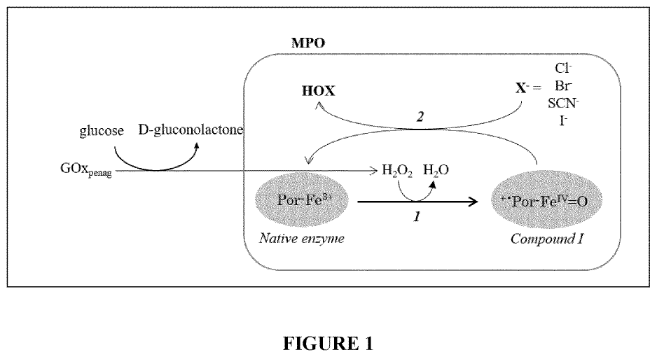

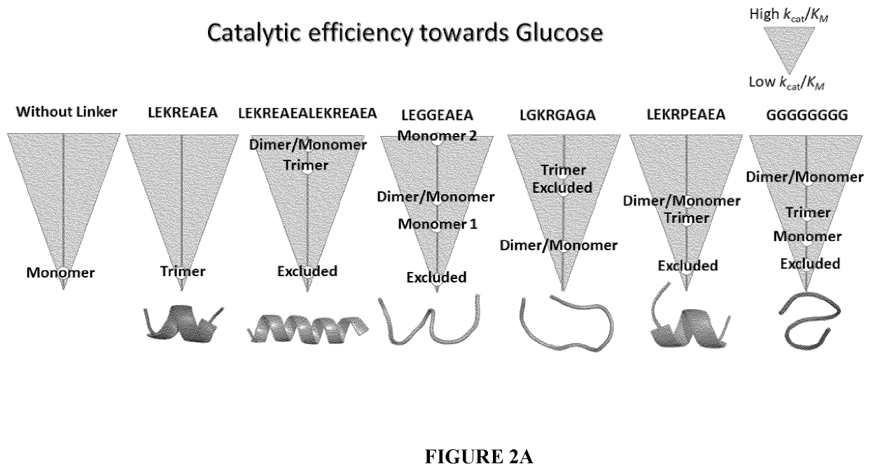

The Megazyme kit was used to measure the production of D-gluconic acid by GOx_{penag} and therefor to solely characterize the specific glucose oxidase activity of the chimeras (FIG. 1). An increase in absorbance at 340 nm indicates the presence of NADPH, and is thus indirect evidence of D-gluconic acid production. The test was performed at pH6, which is known to be optimal for GOx_{penag}. After 17 h of incubation, A_{340 nm} was measured to make a blank. Then, as a last step, the production of NADPH was triggered and an increase in A_{340 nm} was observed (data not shown). Based on the ΔA_{340 nm} values, it was possible to calculate the concentration of D-gluconic acid produced by the chimeras and determine their specific glucose oxidase activity, as shown in Table 7 below.

TABLE 7
Steady-state kinetic parameters of the chimeras (glucose oxidase activity). All
tests were performed at least in triplicate. “±” means Mean Standard Error.

| | Oligomeric | [D-Gluconate] | Enzyme | D-Gluconate | Apparent |
|---|---|---|---|---|---|
| | state | produced in | quantity | (nmol) in 100 | specific GOx |
| RbMPO-GOx chimera | (n ≥ 1) | 17h (g/L) | (μg) | μL Megazyme | activity (IU/mg) |
| No linker | monomer | 0.156 ± 0.0156 | 11.2 | 79.6 ± 7.9 | 6.963E−03 ± 0.696E−03 |
| LEKREAEA | trimer | 5.42 ± 0.542 | 1.1 | 2764 ± 276.4 | 2.463 ± 0.246 |
| LEKRPEAEA | excluded | 1.744 ± 0.06 | 13.2 | 889.5 ± 30.8 | 0.066 ± 0.002 |
| | trimer | 7.8 ± 0.96 | 14.5 | 3978 ± 490 | 0.268 ± 0.033 |
| | dimer/ | 6.871 ± 0.304 | 14.1 | 3504 ± 190 | 0.244 ± 0.013 |
| | monomer | | | | |
| LEKREAEALEKREAEA | excluded | 0.51 ± 0.077 | 16.56 | 289 ± 39 | 0.017 ± 0.002 |
| | trimer | 8.52 ± 0.80 | 13.91 | 4347 ± 430 | 0.306 ± 0.030 |
| | dimer/ | 5.59 ± 0.56 | 13.29 | 2854 ± 285 | 0.210 ± 0.021 |
| | monomer | | | | |
| LEGGEAEA | excluded | 6.920E−03 ± 0.419E−03 | 1.4 | 2.9 ± 0.9 | 1.940E−03 ± 0.664E−03 |
| | dimer/ | 0.45 ± 0.03 | 1.4 | 229.3 ± 15.2 | 0.156 ± 0.01 |
| | monomer | | | | |
| | monomer 1 | 2.348 ± 0.1 | 1.6 | 1197.6 ± 50.8 | 0.731 ± 0.031 |
| | monomer 2 | 0.243 ± 0.01 | 1.4 | 123.8 ± 5.1 | 0.086 ± 0.004 |
| LGKRGAGA | excluded | 0.163 ± 0.01 | 1.1 | 83.1 ± 4.9 | 0.070 ± 0.004 |
| | trimer | 1.117 ± 0.025 | 1.3 | 569.7 ± 12.8 | 0.443 ± 0.01 |
| | dimer/ | 0.748 ± 0.035 | 1.4 | 381.3 ± 17.6 | 0.271 ± 0.013 |
| | monomer | | | | |
| GGGGGGGG | excluded | 0.21 ± 0.007 | 1.4 | 106.9 ± 3.4 | 0.073 ± 0.002 |
| | trimer | 1.307 ± 0.316 | 1.1 | 666.6 ± 161.2 | 0.639 ± 0.155 |
| | dimer/ | 1.119 ± 0.124 | 1.6 | 570.7 ± 63.2 | 0.352 ± 0.039 |
| | monomer | | | | |
| | monomer | 0.056 ± 0.008 | 1.5 | 28.3 ± 3.8 | 0.019 ± 0.003 |
| LEGGEAEA | mixture | 0.849 ± 0.196 | 1.4 | 433.1 ± 70.9 | 0.298 ± 0.049 |

All chimeras displayed glucose oxidase activity, albeit with different efficiency.

The most active chimera was RbMPO-LEKREAEA-GOx (2.463±0.246 IU/mg) followed by monomer of RbMPO-LEGGEAEA-GOx (0.731±0.031 IU/mg), the trimer of RbMPO-GGGGGGGG-GOx (0.639±0.155 IU/mg), the trimer of RbMPO-LGKRGAGA-GOx (0.443±0.01 IU/mg), and finally the trimer of RbMPO-LEKRPEAEA-GOx (0.268±0.03 IU/mg).

Likewise, the chimera RbMPO-LEGGEAEA-GOx produced as a mixture displayed a glucose oxidase activity, since the production of D-gluconic acid from glucose was detected with the Megazym kit, at a level of 0.298±0.049 IU/mg.

### 2.3.2. Myeloperoxidase Activity

### Using Chloride as a Substrate

In order to determine the RbMPO activity of the chimeras, a H₂O₂ concentration range was performed at a fixed concentration of NaCl. HOCl reacted with APF probe and apparition of fluorescein was monitored. Steady-state kinetic parameters are summarized in Table 8 below.

TABLE 8
Steady-state kinetic parameters of the chimeras (chlorination activity). All tests
were performed at least in triplicate. “±” means Mean Standard Error. “n.a” means not
applicable.

| | Oligomeric | | | | |
|---|---|---|---|---|---|
| RbMPO-GOx | state | | K_{M} H₂O₂ | k_{cat}/K_{M} | Kᵢ H₂O₂ |
| chimera | (n ≥ 1) | k_{cat} (s⁻¹) | (mM) | (M⁻¹.s⁻¹) | (mM) |
| No linker | monomer | 1.20E−03 ± | 0.270 ± 0.14 | 4.44 ± 3.15 | 1.910 ± 0.480 |
| | | 0.228E−03 | | | |
| LEKREAEA | trimer | 5.870E−04 ± | 0.045 ± 0.015 | 13.04 ± 6.51 | 2.12 ± 1.70 |
| | | 0.973E−04 | | | |
| LEKRPEAEA | excluded | 1.553E−04 ± | 0.023 ± 0.011 | 6.82 ± 4.27 | 17.3 ± 10.6 |
| | | 0.204E−04 | | | |
| | trimer | 3.000E−04 ± | 0.704 ± 0.448 | 0.426 ± 0.365 | 35.9 ± 22.2 |
| | | 0.655E−04 | | | |
| | dimer/ | 5.76E−04 ± | 0.036 ± 0.025 | 16.08 ± 17.15 | 6.30 ± 3.33 |
| | monomer | 2.11E−04 | | | |
| LEKREAEAL | excluded | n.a. | n.a. | kss/[H₂O₂]: | n.a. |
| EKREAEA | | | | 5.10E−03 ± 0.35E−03 | |
| | trimer | 4.70E−03 ± | 0.013 ± 0.001 | 361.5 ± 64.5 | 0.597 ± 0.211 |
| | | 0.48E−03 | | | |
| | dimer/ | 3.74E−03 ± | 2.48E−03 ± 2.18E−03 | 1508 ± 107 | 4.63 ± 1.05 |
| | monomer | 0.26E−03 | | | |
| LEGGEAEA | monomer 1 | 3.086E−04 ± | 9.02E−03 ± 4.55E−03 | 34.2 ± 19.4 | 34.9 ± 12.6 |
| | | 0.193E−04 | | | |
| GGGGGGGG | excluded | 1.682E−04 ± | 0.168 ± 0.081 | 0.998 ± 0.603 | 140.5 ± 65.6 |
| | | 0.206E−04 | | | |
| | trimer | 5.086E−04 ± | 2.86E−03 ± 1.71E−03 | 177.8 ± 133.1 | 5.92 ± 3.22 |
| | | 0.765E−04 | | | |
| | dimer/ | 4.18E−03 ± | 1.15E−02 ± 0.76E−02 | 363.5 ± 382.3 | 3.29E−02 ± 1.93E−02 |
| | monomer | 1.63E−03 | | | |
| | monomer | 1.798E−04 ± | 4.70E−03 ± 2.03E−03 | 38.3 ± 23.6 | 13.1 ± 4.9 |
| | | 0.329E−04 | | | |

All chimeras displayed a chlorination activity, albeit with different efficiency.

The most active chimera was RbMPO-LEKREAEALEKREAEA-GOx in the dimer/monomer state, followed by its trimer state. The dimer/monomer form of this chimera had indeed a high specificity constant (1508±19.4 M⁻¹·s⁻¹) compared to other chimeras. In terms of catalytic efficiency, these chimeras were followed by RbMPO-LEGGEAEA-GOx (34.2±19.4 M⁻¹·s⁻¹), and RbMPO-LEKRPEAEA-GOx (excluded) (6.82±4.27 M⁻¹·s⁻¹) with a slightly greater inhibition by H₂O₂(Ki=17.3±10.6 mM). RbMPO-GGGGGGGG-GOx (excluded) had a low specificity constant (1±0.6 M⁻¹·s⁻¹) but a very low inhibition by H₂O₂(Ki=140.5±65.6 mM). At last, the chimera RbMPO-GOx devoid of linker had an average specificity constant (4.44±3.15 M⁻¹·s⁻¹).

The chimera RbMPO-LEGGEAEA-GOx produced as a mixture of oligomers also displayed a myeloperoxidase activity, since HOCl with APF was detected. A kss around 10⁻³ s⁻¹ at 15 mM glucose and 500 mM NaCl was determined. Because of the heterogeneous nature of this protein, it was nevertheless not possible to determine the individual kinetic parameters kcat, K_{M} and Kᵢ.

### 2.4. Enzyme Characterization of Coupled Active Sites of the Chimeras

### Using Chloride and Glucose as Substrates

The activity of the two active sites was then assessed in conjunction to each other (FIG. 1).

Briefly, the aim was to measure the production of HOCl from glucose and NaCl as respective substrates of the enzymes, using the APF probe. Two types of kinetics were performed:
i) kinetics at various concentrations of glucose and a fixed concentration of NaCl, and
ii) kinetics at various concentration of NaCl and a fixed concentration of glucose.

Steady-state kinetic parameters are summarized in Tables 9 and 10 below. P

TABLE 9
Steady-state kinetic parameters of the chimeras (glucose oxidase activity). All
tests were performed at least in triplicate. “±” means Mean Standard Error. “n.a.”
means not available (for Kᵢ: chimera not inhibited by an excess of glucose).

| RbMPO-GOx | Oligomeric | | K_{M} glucose | k_{cat}/K_{M} glucose | Kᵢ glucose |
|---|---|---|---|---|---|
| chimera | state (n ≥ 1) | k_{cat} (s⁻¹) | (mM) | (M⁻¹.s⁻¹) | (mM) |
| No linker | monomer | 3.090E−05 ± | 3.02 ± 0.77 | 10.00E−03 ± 3.51E−03 | n.a. |
| | | 0.272E−05 | | | |
| LEKREAEA | trimer | 1.09E−04 ± | 10.62 ± 8.25 | 0.01 ± 0.011 | 84.32 ± 56.47 |
| | | 0.043E−04 | | | |
| LEKRPEAEA | excluded | 3.647E−04 ± | 22.6 ± 6.5 | 0.016 ± 0.005 | 5.70 ± 1.50 |
| | | 0.084E−04 | | | |
| | trimer | 1.560E−04 ± | 1.056 ± 0.496 | 0.148 ± 0.081 | n.a. |
| | | 0.126E−04 | | | |
| | dimer/ | 6.419E−04 ± | 2.16 ± 1.48 | 0.298 ± 0.235 | n.a. |
| | monomer | 0.644E−04 | | | |
| LEKREAEA | excluded | 1.50E−04 ± | 14.17 ± 4.62 | 0.010 ± 0.004 | 20.47 ± 6.38 |
| LEKREAEA | | 0.15E−04 | | | |
| | trimer | 1.37E−03 ± | 0.77 ± 0.50 | 1.77 ± 1.34 | n.a. |
| | | 0.146E−03 | | | |
| | dimer/ | 5.00E−0.3 ± | 2.02 ± 1.07 | 2.47 ± 1.58 | n.a |
| | monomer | 0.55E−03 | | | |
| LEGGEAEA | excluded | 5.600E−04 ± | 65.6 ± 14.2 | 8.53E−03 ± 2.15E−03 | 0.696 ± 0.129 |
| | | 0.203E−04 | | | |
| | dimer/ | 3.150E−04 ± | 0.629 ± 0.295 | 0.5 ± 0.259 | n.a. |
| | monomer | 0.154E−04 | | | |
| | monomer 1 | 1.230E−03 ± | 8.82 ± 4.87 | 0.139 ± 0.095 | n.a. |
| | | 0.158E−03 | | | |
| | monomer 2 | 3.770E−03 ± | 1.38 ± 0.671 | 2.73 ± 1.50 | n.a. |
| | | 0.240E−03 | | | |
| LGKRGAGA | excluded | 1.330E−03 ± | 2.27 ± 1.30 | 0.586 ± 0.384 | n.a. |
| | | 0.109E−03 | | | |
| | trimer | 2.200E−03 ± | 2.022 ± 0.644 | 1.088 ± 0.801 | 6.10 ± 1.27 |
| | | 0.919E−03 | | | |
| | dimer/ | 3.050E−03 ± | 66.9 ± 24.1 | 0.046 ± 0.025 | 619.3 ± 239.3 |
| | monomer | 0.574E−04 | | | |
| GGGGGGGG | excluded | 1.018E−04 ± | 2.76 ± 1.06 | 0.037 ± 0.017 | n.a. |
| | | 0.065E−04 | | | |
| | trimer | 5.89E−04 ± | 3.61 ± 2.11 | 0.163 ± 0.129 | 86.9 ± 43.7 |
| | | 1.24E−04 | | | |
| | dimer/ | 1.550E−03 ± | 2.06 ± 1.29 | 0.754 ± 0.545 | n.a. |
| | monomer | 0.147E−03 | | | |
| | monomer | 5.117E−04 ± | 6.196 ± 0.951 | 0.083 ± 0.015 | n.a. |
| | | 0.166E−04 | | | |

TABLE 10
Steady-state kinetic parameters of the chimeras (chlorination activity). All tests
were performed at least in triplicate. “±” means Mean Standard Error. “n.a.” means not
available (for Kᵢ: chimera not inhibited by an excess of NaCl).

| | Oligomeric | | K_{M} NaCl | k_{cat}/K_{M} | | |
|---|---|---|---|---|---|---|
| | state | | (mM) | NaCl | kss/[NaCl] | Kᵢ NaCl |
| RbMPO-GOx chimera | (n ≥ 1) | k_{cat} (s⁻¹) | | (M⁻¹.s⁻¹) | (M−1.s−1) | (mM) |
| No linker | monomer | n.a. | n.a. | n.a. | 2.71E−05 ± | n.a. |
| | | | | | 1.61E−05 | |
| LEKREAEA | trimer | 1.64E−04 ± | 69.96 ± 44.58 | 2.34E−03 ± | n.a. | n.a. |
| | | 0.335E−04 | | 1.97E−03 | | |
| LEKRPEAEA | excluded | 1.460E−03 ± | 116.2 ± 26.8 | 1.257E−02 ± | n.a. | 4.78 ± |
| | | 0.250E−03 | | 0.506E−02 | | 1.01 |
| | trimer | 2.436E−04 | 16.23 ± 5.82 | 1.503E−02 ± | n.a. | n.a. |
| | | 0.154E−04 | | 0.635E−02 | | |
| | dimer/ | n.a. | n.a. | n.a. | 3.117E−03 ± | n.a. |
| | monomer | | | | 0.172E−03 | |
| LEKREAEAL | excluded | n.a. | n.a. | n.a. | 9.26E−05 ± | n.a. |
| EKREAEA | | | | | 1.51E−05 | |
| | trimer | n.a. | n.a. | n.a. | 1.28E−03 ± | n.a. |
| | | | | | 0.14E−03 | |
| | dimer/ | 5.05E−03 ± | 36.13 ± 17.87 | 0.139 ± | n.a. | n.a. |
| | monomer | 0.52E−03 | | 0.076 | | |
| LEGGEAEA | excluded | n.a. | n.a. | n.a. | 4.456E−05 ± | n.a. |
| | | | | | 0.315E−05 | |
| | dimer/ | 3.529E−04 ± | 35 ± 14.4 | 1.009E−02 ± | n.a. | n.a. |
| | monomer | 0.432E−04 | | 0.538E−02 | | |
| | monomer 1 | n.a. | n.a. | n.a. | 1.482E−03 ± | n.a. |
| | | | | | 0.108E−03 | |
| | monomer 2 | n.a. | n.a. | n.a. | 1.344E−03 ± | n.a. |
| | | | | | 0.078E−03 | |
| LGKRGAGA | excluded | 4.020E−03 ± | 197.5 ± 70.5 | 2.035E−02 ± | n.a. | n.a. |
| | | 0.455E−03 | | 0.957E−02 | | |
| | trimer | n.a. | n.a. | n.a. | 5.296E−03 ± | n.a. |
| | | | | | 0.5782E−03 | |
| | dimer/ | 6.020E−03 ± | 2.492 ± 0.787 | 2.422 ± | n.a. | n.a. |
| | monomer | 0.332E−03 | | 0.896 | | |
| GGGGGGGG | excluded | 3.434E−04 ± | 97.2 ± 39.3 | 3.53E−03 ± | n.a. | n.a. |
| | | 0.358E−04 | | 1.80E−03 | | |
| | trimer | 1.090E−03 ± | 401 ± 300 | 2.72E−03 ± | n.a. | n.a. |
| | | 0.337E−03 | | 2.88E−03 | | |
| | dimer/ | 4.600E−03 ± | 269 ± 146 | 1.71E−02 ± | n.a. | n.a. |
| | monomer | 0.887E−03 | | 1.26E−02 | | |
| | monomer | 6.516E−04 ± | 1.99 ± 1.87 | 3.28E−01 ± | n.a. | n.a. |
| | | 0.375E−04 | | 3.27E−01 | | |

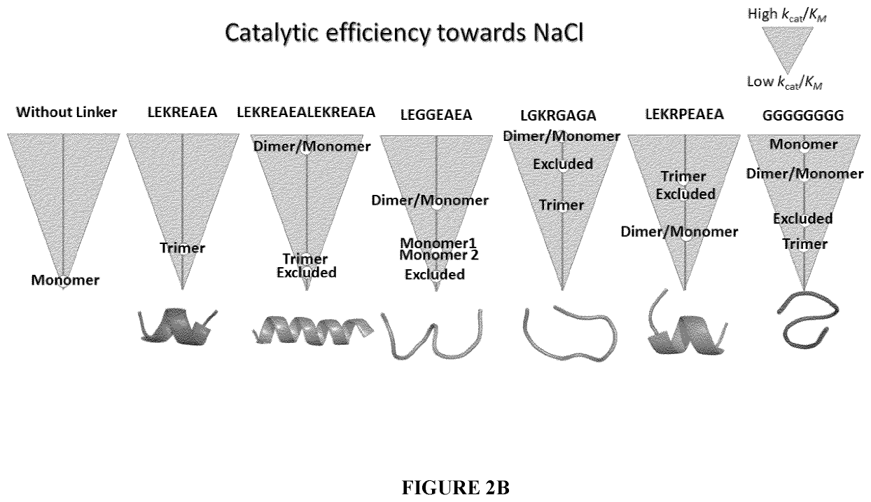

All chimera displayed a glucose activity and a chlorination activity, albeit with different efficiency. The catalytic efficiency of their oligomeric states toward glucose and NaCl is summarized in FIGS. 2A and 2B.

Briefly, with regard to glucose, the monomer 2 of RbMPO-LEGGEAEA-GOx had the best catalytic efficiency (2.73±1.5 M⁻¹·s⁻¹). This chimera was then followed by the dimer/monomer of RbMPO-LEKREAEALEKREAEA-GOx (2.47±1.58 M⁻¹·s⁻¹), the trimer of RbMPO-LEKREAEALEKREAEAGOx (1.7±1.34 M⁻¹·s⁻¹), and the trimer of RbMPO-LGKRGAGA-GOx (1.1±0.8 M⁻¹·s⁻¹) albeit with a strong inhibition (Ki=6.1±1.2 mM). All other chimeras had a kcat/KM below 1 M⁻¹·s⁻¹. Overall, the excluded peaks seemed to have the lowest glucose oxidase activity within each chimera. Most importantly, the presence of a linker improved the catalytic efficiency towards glucose.

With regard to chloride, the dimer/monomer of RbMPO-LGKRGAGA-GOx (2.4±0.9 M⁻¹·s⁻¹) had the highest catalytic efficiency, followed by the dimer/monomer of RbMPO-LEKREAEALEKREAEA-GOx (0.139±0.076 M⁻¹·s⁻¹). These chimeras were followed by the excluded form of RbMPO-LGKRGAGA-GOx (0.020±0.009 M⁻¹·s⁻¹). The other chimeras were also active and presented reactivity below 0.02 M⁻¹·s⁻¹. Because of large error bars, the catalytic efficiencies of the dimer/monomer and trimer of RbMPO-GGGGGGGG-GOx were not conclusive. It shall be further noted that the presence of a linker improved the catalytic efficiency towards chloride.

The chimera RbMPO-LEGGEAEA-GOx produced as a mixture also displayed a glucose oxidase activity combined with a myeloperoxidase activity: the kₛₛ/[Glucose] value for this protein was of 10⁻³ M⁻¹·s⁻¹ when using 15 mM glucose and 500 mM NaCl as initial substrates concentration.

Taken together, these results confirm the positive influence of the peptide linker on the enzymatic catalytic efficiency. Besides, in order to circumvent the variation in catalytic efficiency observed with the different oligomeric states of the chimera, one can use a mixture of these oligomeric states.

### Using Thiocyanate and Glucose as Substrates

For the sake of completion, the RbMPO being capable of catalyzing a wide range of (pseudo)-halides, including not only chloride but also iodide, bromide and thiocyanate (publication in preparation), the myeloperoxidase activity of some of the chimeras was assessed using thiocyanate as a substrate, in presence of a fixed concentration of glucose.

TABLE 11
Steady-state kinetic parameters of the
chimeras (NaSCN as a substrate). All
tests were performed at least in triplicate.
“±” means Mean Standard Error.

| RbMPO-GOx | Oligomeric | | K_{M} SCN | k_{cat}/K_{M} |
|---|---|---|---|---|
| chimera | state | k_{cat} (s⁻¹) | (mM) | (M⁻¹.s⁻¹) |
| LEGGEAEA | monomer 1 | 0.31 ± | 0.220 ± | 1409 ± |
| | | 0.01 | 0.03 | 269 |

These results show that the peptide linker is also suited for efficient catalysis of thiocyanate and glucose.

### 2.5. Microbicidal Activity and Stability of the Chimeras

Microbicidal experiments were then performed with the chimeras that appeared to display the most efficient catalytic efficiencies towards glucose and chloride, i.e.: RbMPO-LEGGEAEA-GOx, RbMPO-LGKRGAGA-GOx, as well as RbMPO-LEKREAEALEKREAEA-GOx. RbMPO-LEGGEAEA-GOx and RbMPO-LGKRGAGA-GOx were also of interest because of their weak inhibition by H₂O2. For the sake of completion, different oligomeric states of these chimeras were assessed, as well as a mixture of oligomeric states (for RbMPO-LEGGEAEA-GOx only).

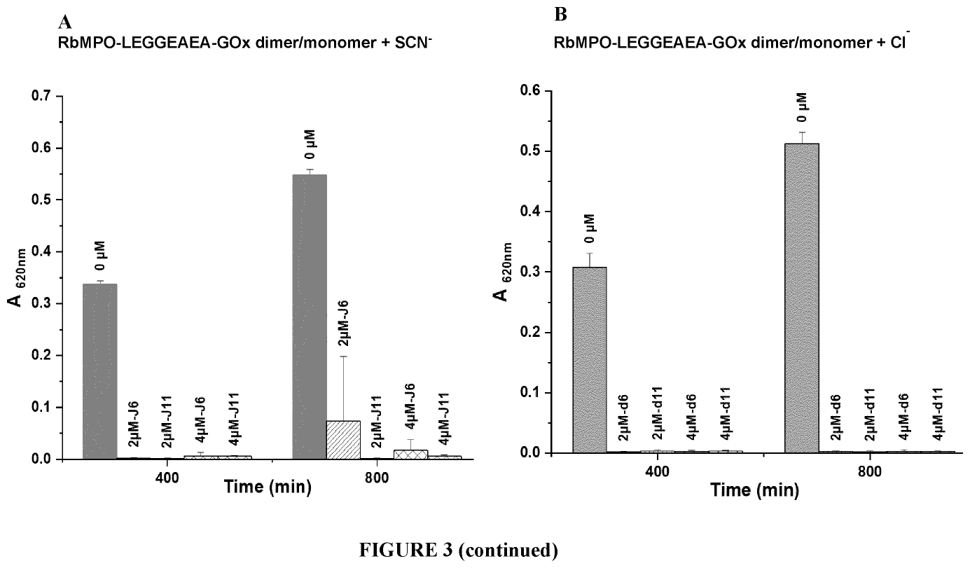

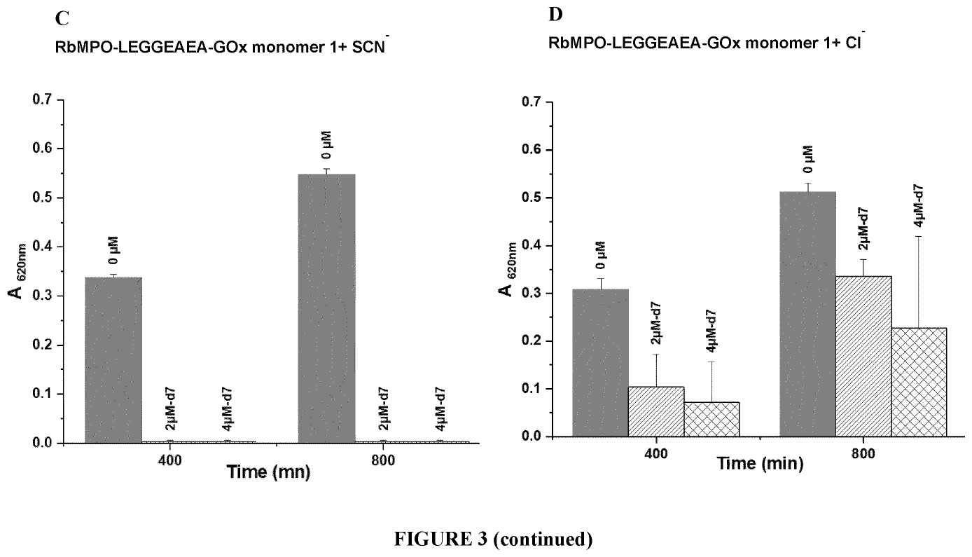

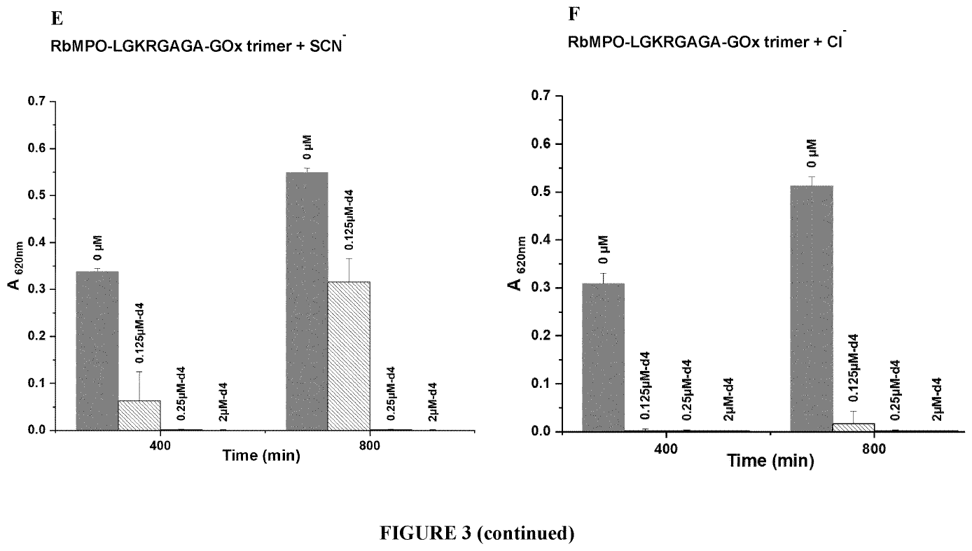

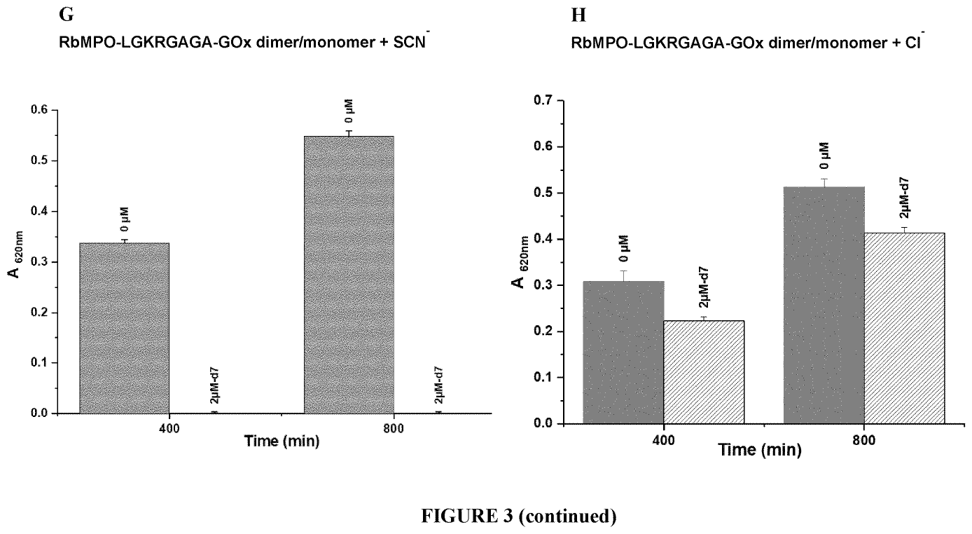

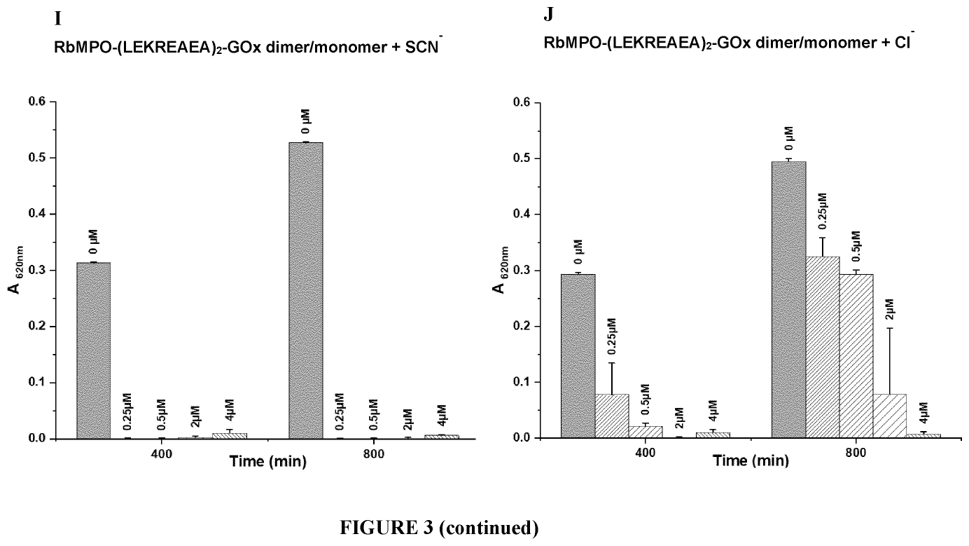

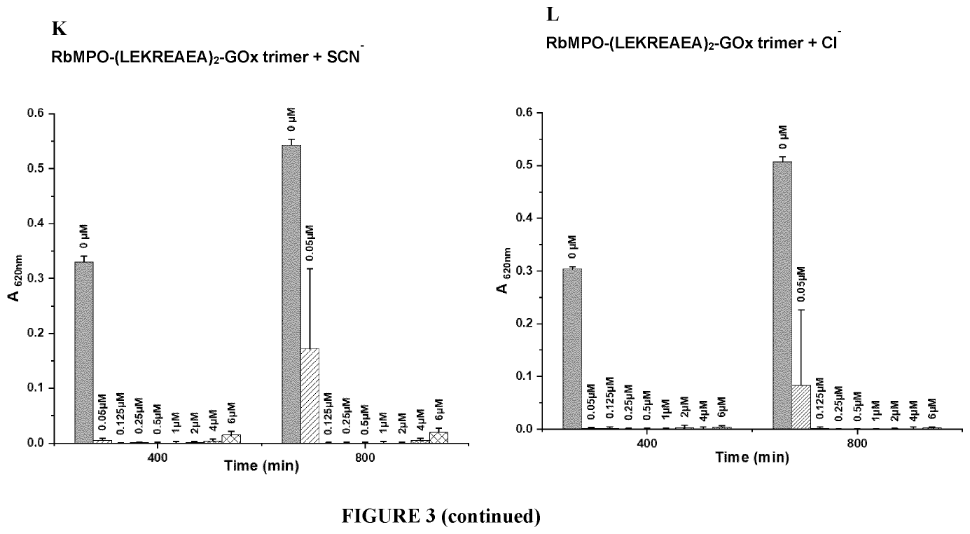

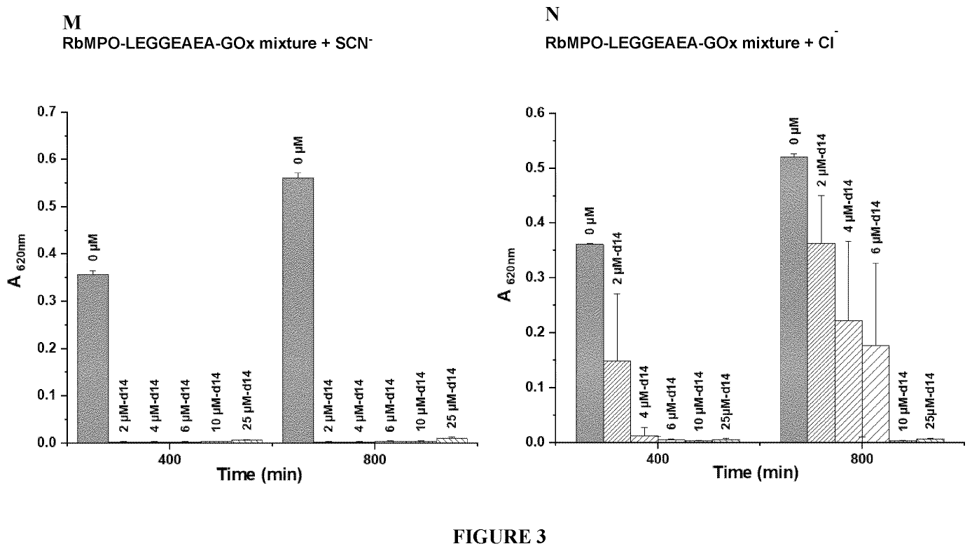

As shown on FIGS. 3A to 3N, all these chimeras inhibited bacterial growth in presence of either NaSCN or NaCl. Interestingly, this microbicidal effect was observed after storage of the chimeras at 4° C. for 4 to 21 days, thereby demonstrating the stability of these chimeras.

### 3. Conclusion

In the present study, various fusions of a GOx and a MPO enzymes were constructed and produced in a suitable bacterial strain. To this end, an 8-day reconstitution step in the presence FAD and heme prosthetic moieties was required. The purification yield was elevated, ranging from about 40 mg (for individual oligomers) to about 604 mg (for the mixture of oligomers) of pure chimeric enzymes per liter of bacterial culture.

A first chimeric protein was produced by fusing the C-terminus of the MPO enzyme directly to the N-terminus of the GOx enzyme.

Different peptide linkers were then genetically engineered between the two enzymes: these linkers display different properties in terms of amino acid charge, amino acid length, amino acid degree of flexibility, and/or tertiary structure. To this end, chimeras were generated with the peptide linker LEKREAEA, or with mutated variants thereof (a proline introduced in the middle of the peptide; one or more positively or negatively charged amino acids mutated into the neutral glycine; all amino acids mutated into the neutral glycine; repetition of the peptide linker). Several oligomeric states (n≥1) of each of these chimeras were identified and purified for further analysis. One chimera was also produced as a mixture of its oligomeric states, so as to reduce the cost and time of production.

The presence of the peptide linker improved the enzymatic activity compared to the chimera devoid of linker, especially at the level of the GOx activity, and also towards NaCl (chlorination activity, which is a myeloperoxidase activity).

Microbicidal property of the most active chimeras, was then investigated: the study confirmed their microbicidal activity, as well as their stability, either in the form of independent oligomeric state or of a mixture thereof.

### Example 2

Experiments similar to those conduced in EXAMPLE 1 were performed so as create chimeric constructions of the myeloperoxidase from Homo sapiens fused to the glucose oxidase from Penicillium amagasakiens. Again, two types of chimeras were obtained by genetic engineering: one with an original peptide linker, and one without such linker between the two enzymes.

1. A non-naturally occurring polypeptide having a myeloperoxidase activity and a glucose oxidase activity.

2. The polypeptide according to claim 1, wherein the polypeptide is a fusion polypeptide comprising a myeloperoxidase coupled, preferably covalently coupled, to a glucose oxidase.

3. The polypeptide according to claim 2, wherein the C-terminus of the myeloperoxidase is coupled, preferably covalently coupled, to the N-terminus of the glucose oxidase.

4. The polypeptide according to claim 2 or 3, wherein the myeloperoxidase is coupled, preferably covalently coupled, to the glucose oxidase by a linker, preferably by a peptide linker.

5. The polypeptide according to claim 4, wherein the linker is peptide linker comprising, or consisting of, the following amino acid sequence: 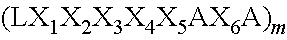
wherein
X₁ is glutamate or glycine,
X₂ is lysine or glycine,
X₃ is arginine or glycine,
X₄ is proline or vacant,
X₅ is glutamate or glycine,
X₆ is glutamate or glycine,
and m is an integer ranging from 1 to 2;
or is a substantially homologous peptide thereof, preferably deriving from said sequence by one or more conservative substitutions.

6. The polypeptide according to claim 5, wherein the peptide linker comprises, or consists, of any one of the following amino acid sequences: LEGGEAEA (SEQ ID NO: 4), LGKRGAGA (SEQ ID NO: 5), (LEKREAEA)₂ (SEQ ID NO: 6), LEKREAEA (SEQ ID NO: 7) or LEKRPEAEA (SEQ ID NO: 8); or is a substantially homologous peptide thereof, preferably deriving from any one of SEQ ID NO: 4 to 8 by one or more conservative substitutions.

7. The polypeptide according to claim 4, wherein the linker is a peptide linker comprising, or consisting of, a polyglycine amino acid sequence; or is a substantially homologous peptide thereof, preferably deriving from said sequence by one or more conservative substitutions.

8. The polypeptide according to any one of the preceding claims, wherein the myeloperoxidase is a microbial myeloperoxidase, preferably a myeloperoxidase from Rhodopirellula baltica, or is a mammalian myeloperoxidase, preferably a myeloperoxidase from Homo sapiens.

9. The polypeptide according to any one of the preceding claims, wherein the glucose oxidase is a microbial glucose oxidase enzyme, preferably a glucose oxidase from Penicillium amagasakiens.

10. The polypeptide according to any one of the preceding claims, wherein the polypeptide is in the form of a functional oligomer, or a mixture of functional oligomers.

11. A nucleic acid encoding a polypeptide as defined in any one of the preceding claims.

12. A vector comprising a nucleic acid as defined in claim 11.

13. A host cell comprising a vector as defined in claim 12.

14. A method for obtaining a polypeptide as defined in any one of claims 1 to 10 comprising at least the steps of:
a) culturing in a medium a host cell as defined in claim 13, under conditions suitable for the expression of the polypeptide; and
b) recovering said polypeptide.

15. The method according to claim 14, wherein the polypeptide recovered in step b) is further solubilized in a solution comprising heme and flavine adenine dinucleotide (FAD), and optionally calcium.

16. An antibacterial composition comprising the non-naturally occurring polypeptide as defined in any one of the claims 1 to 10.

17. The composition according to claim 16, further comprising glucose or a source of glucose and/or a halide or pseudohalide.

18. In vitro use of a non-naturally occurring polypeptide as defined in any one of claims 1 to 10 or of a composition as defined in claim 16 or 17, for halogenating a non-halogenated organic compound.

19. In vitro or ex vivo use of a non-naturally occurring polypeptide as defined in any one of claims 1 to 10 or of a composition as defined in claim 16 or 17, for killing or inhibiting the growth of microorganisms.

20. A non-naturally occurring polypeptide as defined in any one of claims 1 to 10 or a composition as defined in claim 16 or 17, for use as a medicament, preferably for the treatment of a microbial infection.

21. (i) A non-naturally occurring polypeptide as defined in any one of claims 1 to 10 or a composition as defined in claim 16 or 17 and (ii) glucose or a source of glucose, and/or a halide or pseudohalide, as a combined preparation for simultaneous, separate or sequential use as a medicament, preferably for the treatment of a microbial infection.

22. A peptide linker as defined in claim 5 or 6.


SEQUENCE LISTING (26 sequences)

SEQ ID NO: 1 - protein, 7 aa, synthetic construct
  REPEAT 2..6: note = number of repeat is an integer ranging from 2 to 5
AEAAAKA

SEQ ID NO: 2 - protein, 5 aa, synthetic construct
PAPAP

SEQ ID NO: 3 - protein, 5 aa, Synthetic construct
  REPEAT 1..5: note = number of repeat is an integer ranging from 2 to 5
GGGGS

SEQ ID NO: 4 - protein, 8 aa, synthetic construct
LEGGEAEA

SEQ ID NO: 5 - protein, 8 aa, synthetic construct
LGKRGAGA

SEQ ID NO: 6 - protein, 16 aa, synthetic construct
LEKREAEALEKREAEA

SEQ ID NO: 7 - protein, 8 aa, synthetic construct
LEKREAEA

SEQ ID NO: 8 - protein, 9 aa, synthetic construct
LEKRPEAEA

SEQ ID NO: 9 - protein, 8 aa, synthetic construct
GGGGGGGG

SEQ ID NO: 10 - protein, 713 aa, Rhodopirellula baltica
MLFWSKRTRKHPGRKRRRLRSEQLESRQLLAADFGGYRHNIYDAEDVNDDGRVSAIDALLVINAMTSETSRDENVFTDVNGDGRRSALDALRVINRIERGDVFPIDTSGDEDLNTLLPEMPSEVRSIDGSGNNPNDHLWGSVGQRLIRMAPAAYGDGISTLAGANRPSAREVSNVLSAMNTSQVLNDRGLSAFVYVWGQFIDHDLGLTESDEHGEAIDISVPEGDLWFDPMGSGEAVIPMTRTPIADGTGTSVGNPAQQFNQITAFIDGSMVYGSDAATAERLRTFAGGRLAISDNGLLPMDESGMVIAGDVRASENVGLTAIQTLFVREHNRLADEIFAADPEATDEEIYQRARLVVASLIQSITYNEFLPALLGQRAMDSYRGYDASVNPGIANEFSTAAFRLGHSTLRDEVGFMSNDGRESKDEMELKDAFFHASMLEETGIDSLLKFDASVQAQEIDLAVVDSLRNFLFGPPGAGGLDLVAMNIQRGRDHGLSDYNTTRQAYGLDRVETFDQITGDVELQQKLASLYGTVDNIDLWVGLMAEDHQHDASVGELTGLIIADQFQRTRDGDRFFYKNVLTDSEVESIERSTLADLIERNTSVDGLQENVFFMQVELRGRVILASSIPPNTMPSEVREEEIRGVSGMAIELIDASGKLVDSTVSDQHGNYRFRALSETGDYQVRLSESGEAMDVLVAHGAERIRGLDFIVLA

SEQ ID NO: 11 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 12 - protein, 587 aa, Penicillium amagasakiens
YLPAQQIDVQSSLLSDPSKVAGKTYDYIIAGGGLTGLTVAAKLTENPKIKVLVIEKGFYESNDGAIIEDPNAYGQIFGTTVDQNYLTVPLINNRTNNIKAGKGLGGSTLINGDSWTRPDKVQIDSWEKVFGMEGWNWDNMFEYMKKAEAARTPTAAQLAAGHSFNATCHGTNGTVQSGARDNGQPWSPIMKALMNTVSALGVPVQQDFLCGHPRGVSMIMNNLDENQVRVDAARAWLLPNYQRSNLEILTGQMVGKVLFKQTASGPQAVGVNFGTNKAVNFDVFAKHEVLLAAGSAISPLILEYSGIGLKSVLDQANVTQLLDLPVGINMQDQTTTTVSSRASSAGAGQGQAVFFANFTETFGDYAPQARDLLNTKLDQWAEETVARGGFHNVTALKVQYENYRNWLLDEDVAFAELFMDTEGKINFDLWDLIPFTRGSVHILSSDPYLWQFANDPKFFLNEFDLLGQAAASKLARDLTSQGAMKEYFAGETLPGYNLVQNATLSQWSDYVLQNFRPNWHAVSSCSMMSRELGGVVDATAKVYGTQGLRVIDGSIPPTQVSSHVMTIFYGMALKVADAILDDYAKSA

SEQ ID NO: 13 - DNA, 39 bp, synthetic construct
  source 1..39: mol_type = other DNA
agcgaggacgataaaatccaggccgcgaattctttccgc

SEQ ID NO: 14 - DNA, 35 bp, synthetic construct
  source 1..35: mol_type = other DNA
tacctgcctgcccaacagattgatgtccagtctag

SEQ ID NO: 15 - DNA, 29 bp, synthetic construct
  source 1..29: mol_type = other DNA
ggtgccaaagatttctccataagcatttg

SEQ ID NO: 16 - DNA, 36 bp, synthetic construct
  source 1..36: mol_type = other DNA
ccgccgcgtttcggttgggggcgagcacgcttcgtg

SEQ ID NO: 17 - DNA, 37 bp, synthetic construct
  source 1..37: mol_type = other DNA
ggcggcggcggctacctgcctgcccaacagattgatg

SEQ ID NO: 18 - DNA, 38 bp, synthetic construct
  source 1..38: mol_type = other DNA
gccgccgccgccagcgaggacgataaaatccaggccgc

SEQ ID NO: 19 - DNA, 37 bp, synthetic construct
  source 1..37: mol_type = other DNA
ggcgccggcgcctacctgcctgcccaacagattgatg

SEQ ID NO: 20 - DNA, 38 bp, synthetic construct
  source 1..38: mol_type = other DNA
tcttttgccgagagcgaggacgataaaatccaggccgc

SEQ ID NO: 21 - DNA, 38 bp, synthetic construct
  source 1..38: mol_type = other DNA
gagggctgaagcttacctgcctgcccaacagattgatg

SEQ ID NO: 22 - DNA, 43 bp, synthetic construct
  source 1..43: mol_type = other DNA
tcttttctcgagagcttcagcctctcttttctcgagagcgagg

SEQ ID NO: 23 - DNA, 38 bp, synthetic construct
  source 1..38: mol_type = other DNA
cgtcctcgctctcgagggcggcgaggctgaagcttacc

SEQ ID NO: 24 - DNA, 38 bp, synthetic construct
  source 1..38: mol_type = other DNA
ggtaagcttcagcctcgccgccctcgagagcgaggacg

SEQ ID NO: 25 - DNA, 44 bp, synthetic construct
  source 1..44: mol_type = other DNA
cgtcctcgctctcgagaaaagaccggaggctgaagcttcctgaa

SEQ ID NO: 26 - DNA, 45 bp, synthetic construct
  source 1..45: mol_type = other DNA
ggcaggtaagcttcagcctccggtcttttctcgagagcgaggacg